(19) United States
(12) Patent Application Publication
(10) Pub. No.: US 2024/0189414 A1
(43) Pub. Date: Jun. 13, 2024

(54) ENGINEERED HEARTLAND VIRUS MRNA VACCINE

(71) Applicants: Vernagen, LLC, Tucker, GA (US); The United States of America, as represented by the Secretary, Department of Health and Human Servic, Bethesda, MD (US)

(72) Inventors: Baek KIM, Atlanta, GA (US); Jack Yoon, Suwanee, GA (US); David Pak, Suwanee, GA (US); Claire Yuhui Huang Kinney, Fort Collins, CO (US)

(73) Assignees: Vernagen, LLC, Tucker, GA (US); The United States of America, as represented by the Secretary, Department of Health and Human Servic, Bethesda, MD (US)

(21) Appl. No.: 18/530,861

(22) Filed: Dec. 6, 2023

Related U.S. Application Data
(60) Provisional application No. 63/432,266, filed on Dec. 13, 2022.

(51) Int. Cl. A61K 39/12; A61K 9/51; C12N 7/00

(52) U.S. Cl. CPC A61K 39/12; A61K 9/5123; C12N 7/00

(57) ABSTRACT

Provided herein are a Heartland virus vaccine composition including a messenger ribonucleic acid (mRNA) including an open reading frame (ORF) encoding Gn or Gc of Heartland virus, a Heartland virus vaccine composition comprising a messenger ribonucleic acid (mRNA) comprising an open reading frame (ORF) encoding Gn or Gc of Heartland virus fused with human collagen type I alpha 1 (COL1A1) signal peptide, and a method of inducing immune response against Heartland virus by administering an effective amount of the Heartland virus vaccine composition to a subject in need thereof.

### CROSS REFERENCE TO RELATED APPLICATIONS

This application claims priority to U.S. Provisional Application No. 63/432,266 filed Dec. 13, 2022, the entire disclosure of which is incorporated herein by reference.

### INCORPORATION BY REFERENCE OF SEQUENCE LISTING

The content of the electronically submitted sequence listing, file name: A293206_sequence listing as filed; size: 48,873 bytes; and date of creation: Nov. 13, 2023, filed herewith, is incorporated herein by reference in its entirety.

### FIELD

Provided herein are a Heartland virus vaccine composition comprising a messenger ribonucleic acid (mRNA) comprising an open reading frame (ORF) encoding Gn or Gc of Heartland virus, a Heartland virus vaccine composition comprising a messenger ribonucleic acid (mRNA) comprising an open reading frame (ORF) encoding Gn or Gc of Heartland virus fused with human collagen type I alpha 1 (COL1A1) signal peptide, and a method of inducing immune response against Heartland virus by administering an effective amount of the Heartland virus vaccine composition to a subject in need thereof.

### BACKGROUND

Heartland virus, also known as Heartland bandavirus, is a tick-borne phlebovirus of the Bhanja virus serocomplex. At present, there is no approved Heartland virus mRNA vaccine, and there has been a need for Heartland virus mRNA vaccine.

### SUMMARY

The present disclosure provides a Heartland virus vaccine composition comprising a messenger ribonucleic acid (mRNA) comprising an open reading frame (ORF) encoding Gn or Gc of Heartland virus, or the Gn or Gc of Heartland virus fused with human collagen type I alpha 1 (COL1A1) signal peptide. In one embodiment, the Gn of Heartland virus has an amino acid sequence of SEQ ID NO: 1. In another embodiment, the Gc of Heartland virus has an amino acid sequence of SEQ ID NO: 2. In some embodiment, the Gn of Heartland virus fused with COL1A1 signal peptide has an amino acid sequence of SEQ ID NO: 3. In one embodiment, the Gc of Heartland virus fused with COL1A1 signal peptide has an amino acid sequence of SEQ ID NO: 4. In some embodiment, the ORF encoding Gn of Heartland virus has a nucleotide sequence of SEQ ID NO: 5. In another embodiment, the ORF encoding Gc of Heartland virus has a nucleotide sequence of SEQ ID NO: 6. In one embodiment, the ORF encoding Gn of Heartland virus fused with COL1A1 signal peptide has a nucleotide sequence of SEQ ID NO: 7. In some embodiment, the ORF encoding Gc of Heartland virus fused with COL1A1 signal peptide has a nucleotide sequence of SEQ ID NO: 8. In another embodiment, the mRNA comprising the ORF encoding Gn of Heartland virus further comprises a 5′ untranslated region (UTR), a 3′ UTR, and a poly (A) tail so as to have the following structure of 5′UTR-ORF encoding Gn of Heartland virus-3′UTR-poly (A) tail, and the ORF encoding Gn of Heartland virus has a nucleotide sequence of SEQ ID NO: 5. In some embodiment, the mRNA comprising the ORF encoding Gc of Heartland virus further comprises a 5′ untranslated region (UTR), a 3′ UTR, and a poly (A) tail so as to have the following structure of 5′UTR-ORF encoding Gc of Heartland virus-3′UTR-poly (A) tail, and the ORF encoding Gc of Heartland virus has a nucleotide sequence of SEQ ID NO: 6. In another embodiment, the mRNA comprising the ORF encoding Gn of Heartland virus fused with COL1A1 signal peptide further comprises a 5′ untranslated region (UTR), a 3′ UTR, and a poly (A) tail so as to have the following structure of 5′UTR-ORF encoding Gn of Heartland virus fused with COL1A1 signal peptide-3′UTR-poly (A) tail, and the ORF encoding Gn of Heartland virus fused with COL1A1 signal peptide has a nucleotide sequence of SEQ ID NO: 7. In one embodiment, the mRNA comprising the ORF encoding Gc of Heartland virus fused with COL1A1 signal peptide further comprises a 5′ untranslated region (UTR), a 3′ UTR, and a poly (A) tail so as to have the following structure of 5′UTR-ORF encoding Gc of Heartland virus fused with COL1A1 signal peptide-3′UTR-poly (A) tail, and the ORF encoding Gc of Heartland virus fused with COL1A1 signal peptide has a nucleotide sequence of SEQ ID NO: 8. In some embodiment, the poly (A) tail has a length of 50-250 nucleotides. In one embodiment, the poly (A) tail has a length of 50-250 nucleotides. In some embodiment, the poly (A) tail has a length of 50-250 nucleotides. In another embodiment, the poly (A) tail has a length of 50-250 nucleotides. In one embodiment, the mRNA having the structure of 5′UTR-ORF encoding Gn of Heartland virus-3′UTR-poly (A) tail has a nucleotide sequence of SEQ ID NO: 9. In some embodiment, the mRNA having the structure of 5′UTR-ORF encoding Gc of Heartland virus-3′UTR-poly (A) tail has a nucleotide sequence of SEQ ID NO: 10. In another embodiment, the mRNA having the structure of 5′UTR-ORF encoding Gn of Heartland virus fused with COL1A1 signal peptide-3′UTR-poly (A) tail has a nucleotide sequence of SEQ ID NO: 11. In some embodiment, the mRNA having the structure of 5′UTR-ORF encoding Gc of Heartland virus fused with COL1A1 signal peptide-3′UTR-poly (A) tail has a nucleotide sequence of SEQ ID NO: 12. In another embodiment, the mRNA having the structure of 5′UTR-ORF encoding Gn of Heartland virus-3′UTR-poly (A) tail has a nucleotide sequence having at least 80% identity to SEQ ID NO: 9. In some embodiment, the mRNA having the structure of 5′UTR-ORF encoding Gc of Heartland virus-3′UTR-poly (A) tail has a nucleotide sequence having at least 80% identity to SEQ ID NO: 10. In one embodiment, the mRNA having the structure of 5′UTR-ORF encoding Gn of Heartland virus fused with COL1A1 signal peptide-3′UTR-poly (A) tail has a nucleotide sequence having at least 80% identity to SEQ ID NO: 11. In another embodiment, the mRNA having the structure of 5′UTR-ORF encoding Gc of Heartland virus fused with COL1A1 signal peptide-3′UTR-poly (A) tail has a nucleotide sequence having at least 80% identity to SEQ ID NO: 12. In some embodiment, the Heartland virus vaccine composition according to the present disclosure further comprises a pharmaceutically acceptable carrier. In one embodiment, the pharmaceutically acceptable carrier is a lipid nanoparticle encapsulating the mRNA therein.

The present disclosure also provides a method of inducing immune response against Heartland virus comprising administering an effective amount of the Heartland virus vaccine composition according to the present disclosure to a subject in need thereof.

### BRIEF DESCRIPTION OF THE DRAWINGS

FIG. 1 shows in vitro transcription for Gn of Heartland virus mRNA, Gc of Heartland virus mRNA, Gn of Heartland virus fused with COL1A1 signal peptide mRNA, and Gc of Heartland virus fused with COL1A1 signal peptide mRNA.

FIG. 2A shows the results of western blot from mRNA transfection.

FIG. 2B shows the results of western blot from NLP formulation.

FIG. 3 shows HRTV mRNA vaccination scheme in mice.

FIG. 4A shows survival rate after HRTV challenge.

FIG. 4B shows weight loss after HRTV challenge.

FIG. 5 shows immunogenicity for neutralizing antibody (PRNT50).

FIG. 6 shows viremia at post lethal HRTV challenge.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “Heartland virus vaccine composition” refers to a substance used to stimulate the production of antibodies and provide immunity against Heartland virus.

As used herein, the term “messenger ribonucleic acid (mRNA)” refers to a single-stranded molecule of RNA that corresponds to the genetic sequence of a gene, and is read by a ribosome in the process of synthesizing a protein.

As used herein, the term “fused with” refers to a gene or gene product which has the characteristics of that gene or gene product when isolated from a naturally occurring source.

The term “Gn or Gc of Heartland virus fused with human collagen type I alpha 1 (COL1A1) signal peptide” refers to a recombinant fusion protein created through genetic engineering of a fusion gene. For instance, this may involve removing the stop codon from a cDNA sequence coding for Gn or Gc of Heartland virus, then appending the cDNA sequence of COL1A1 signal peptide in frame through ligation or overlap extension PCR.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “open reading frame (ORF)” refers to a nucleotide sequence between the start and stop codons.

As used herein, the term “an open reading frame (ORF) encoding” refers to the nucleotide coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

As used herein, the term “T7 promoter” refers to a promoter derived from a bacteriophage T7.

As used herein, the term “5′ untranslated region (UTR)” refers to a region of an mRNA that is directly upstream (i.e., 5′) from the start codon (the first codon of an mRNA transcript translated by a ribosome) that does not encode a polypeptide.

As used herein, the term “3′ untranslated region (UTR)” refers to a region of an mRNA that is directly downstream (i.e., 3′) from the stop codon (i.e., the codon of an mRNA transcript that signals a termination of translation) that does not encode a polypeptide.

As used herein, the term “poly (A) tail” refers to a long stretch of adenine nucleotides added to the “tail” or 3′ end of the mRNA.

As used herein, the term “pharmaceutically acceptable carrier” refers to any substance or vehicle suitable for delivering a mRNA vaccine to a suitable in vivo or ex vivo site. Such a carrier can include, but is not limited to, an adjuvant, an excipient, a lipid particle, etc.

As used herein, the term “lipid nanoparticle” refers to a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

As used herein, the term “inducing immune response against Heartland virus” refers to providing protective immunity and/or vaccinating a subject against Heartland virus for prophylactic purposes, as well as causing a desired immune response or effect in a subject in need thereof against Heartland virus, for therapeutic purposes. As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all.

An “effective amount” of the Heartland virus vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the Heartland virus vaccine (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

As used herein, the term “X % identity to SEQ ID NO: Y” or “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “nucleotide sequence having at least X % identity to SEQ ID NO: Y and encodes Z protein” means that the nucleotide sequence meets the two different requirements of having at least X % identity to SEQ ID NO: Y and encoding Z protein. As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The terms “subject,” “patient,” “individual,” and the like are used interchangeably herein, and refer to any animal, any mammalian subject, or cells thereof whether in vitro or in situ, amenable to the methods described herein. In certain non-limiting embodiments, the patient, subject or individual is a human.

### DETAILED DESCRIPTION

### 1. The Heartland Virus Vaccine Composition

The present disclosure provides a Heartland virus vaccine composition comprising a messenger ribonucleic acid (mRNA) comprising an open reading frame (ORF) encoding Gn or Gc of Heartland virus, or the Gn or Gc of Heartland virus fused with human collagen type I alpha 1 (COL1A1) signal peptide.

In one embodiment, the Gn of Heartland virus has an amino acid sequence of SEQ ID NO: 1. In another embodiment, the Gc of Heartland virus has an amino acid sequence of SEQ ID NO: 2. In one embodiment, the Gn of Heartland virus fused with COL1A1 signal peptide has an amino acid sequence of SEQ ID NO: 3. In another embodiment, the Gc of Heartland virus fused with COL1A1 signal peptide has an amino acid sequence of SEQ ID NO: 4.

In one embodiment, the Gn of Heartland virus has an amino acid sequence having at least 80% identity to SEQ ID NO: 1. In another embodiment, the Gn of Heartland virus has an amino acid sequence having at least 85% identity to SEQ ID NO: 1. In some embodiment, the Gn of Heartland virus has an amino acid sequence having at least 90% identity to SEQ ID NO: 1. In another embodiment, the Gn of Heartland virus has an amino acid sequence having at least 95% identity to SEQ ID NO: 1. In one embodiment, the Gn of Heartland virus has an amino acid sequence having at least 96% identity to SEQ ID NO: 1. In some embodiment, the Gn of Heartland virus has an amino acid sequence having at least 97% identity to SEQ ID NO: 1. In another embodiment, the Gn of Heartland virus has an amino acid sequence having at least 98% identity to SEQ ID NO: 1. In some embodiment, the Gn of Heartland virus has an amino acid sequence having at least 99% identity to SEQ ID NO: 1.

In another embodiment, the Gc of Heartland virus has an amino acid sequence having at least 80% identity to SEQ ID NO: 2. In some embodiment, the Gc of Heartland virus has an amino acid sequence having at least 85% identity to SEQ ID NO: 2. In one embodiment, the Gc of Heartland virus has an amino acid sequence having at least 90% identity to SEQ ID NO: 2. In some embodiment, the Gc of Heartland virus has an amino acid sequence having at least 95% identity to SEQ ID NO: 2. In another embodiment, the Gc of Heartland virus has an amino acid sequence having at least 96% identity to SEQ ID NO: 2. In some embodiment, the Gc of Heartland virus has an amino acid sequence having at least 97% identity to SEQ ID NO: 2. In another embodiment, the Gc of Heartland virus has an amino acid sequence having at least 98% identity to SEQ ID NO: 2. In some embodiment, the Gc of Heartland virus has an amino acid sequence having at least 99% identity to SEQ ID NO: 2.

In one embodiment, the Gn of Heartland virus fused with COL1A1 signal peptide has an amino acid sequence having at least 80% identity to SEQ ID NO: 3. In some embodiment, the Gn of Heartland virus fused with COL1A1 signal peptide has an amino acid sequence having at least 85% identity to SEQ ID NO: 3. In another embodiment, the Gn of Heartland virus fused with COL1A1 signal peptide has an amino acid sequence having at least 90% identity to SEQ ID NO: 3. In some embodiment, the Gn of Heartland virus fused with COL1A1 signal peptide has an amino acid sequence having at least 95% identity to SEQ ID NO: 3. In another embodiment, the Gn of Heartland virus fused with COL1A1 signal peptide has an amino acid sequence having at least 96% identity to SEQ ID NO: 3. In some embodiment, the Gn of Heartland virus fused with COL1A1 signal peptide has an amino acid sequence having at least 97% identity to SEQ ID NO: 3. In another embodiment, the Gn of Heartland virus fused with COL1A1 signal peptide has an amino acid sequence having at least 98% identity to SEQ ID NO: 3. In some embodiment, the Gn of Heartland virus fused with COL1A1 signal peptide has an amino acid sequence having at least 99% identity to SEQ ID NO: 3.

In another embodiment, the Gc of Heartland virus fused with COL1A1 signal peptide has an amino acid sequence having at least 80% identity to SEQ ID NO: 4. In one embodiment, the Gc of Heartland virus fused with COL1A1 signal peptide has an amino acid sequence having at least 85% identity to SEQ ID NO: 4. In some embodiment, the Gc of Heartland virus fused with COL1A1 signal peptide has an amino acid sequence having at least 90% identity to SEQ ID NO: 4. In another embodiment, the Gc of Heartland virus fused with COL1A1 signal peptide has an amino acid sequence having at least 95% identity to SEQ ID NO: 4. In some embodiment, the Gc of Heartland virus fused with COL1A1 signal peptide has an amino acid sequence having at least 96% identity to SEQ ID NO: 4. In another embodiment, the Gc of Heartland virus fused with COL1A1 signal peptide has an amino acid sequence having at least 97% identity to SEQ ID NO: 4. In some embodiment, the Gc of Heartland virus fused with COL1A1 signal peptide has an amino acid sequence having at least 98% identity to SEQ ID NO: 4. In one embodiment, the Gc of Heartland virus fused with COL1A1 signal peptide has an amino acid sequence having at least 99% identity to SEQ ID NO: 4.

The present disclosure provides four different types of Heartland virus vaccine compositions as follows.
Heartland virus vaccine composition (1): a Heartland virus vaccine composition comprising a mRNA comprising an ORF encoding Gn of Heartland virus
Heartland virus vaccine composition (2): a Heartland virus vaccine composition comprising a mRNA comprising an ORF encoding Gc of Heartland virus
Heartland virus vaccine composition (3): a Heartland virus vaccine composition comprising a mRNA comprising an ORF encoding Gn of Heartland virus fused with COL1A1 signal peptide
Heartland virus composition (4): a Heartland virus vaccine composition comprising a mRNA comprising an ORF encoding Gc of Heartland virus fused with COL1A1 signal peptide

In the above four types of the Heartland virus vaccine compositions, the Gn of Heartland virus may have an amino acid sequence of SEQ ID NO: 1 (or an amino acid sequence having at least 80%, 85%, 90%, 95%, 96%, 97%, 98%, 99% or 99% identity to SEQ ID NO: 1). In another embodiment, the Gc of Heartland virus may have an amino acid sequence of SEQ ID NO: 2 (or an amino acid sequence having at least 80%, 85%, 90%, 95%, 96%, 97%, 98%, 99% or 99% identity to SEQ ID NO: 2). In one embodiment, the Gn of Heartland virus fused with COL1A1 signal peptide may have an amino acid sequence of SEQ ID NO: 3 (or an amino acid sequence having at least 80%, 85%, 90%, 95%, 96%, 97%, 98%, 99% or 99% identity to SEQ ID NO: 3). In another embodiment, Gc of Heartland virus fused with COL1A1 signal peptide may have an amino acid sequence of SEQ ID NO: 4 (or an amino acid sequence having at least 80%, 85%, 90%, 95%, 96%, 97%, 98%, 99% or 99% identity to SEQ ID NO: 4).

In the above four types of the Heartland virus vaccine compositions, the ORF encoding Gn of Heartland virus may have a nucleotide sequence of SEQ ID NO: 5 (or a nucleotide sequence having at least 80%, 85%, 90%, 95%, 96%, 97%, 98%, 99% or 99% identity to SEQ ID NO: 5). In another embodiment, the ORF encoding Gc of Heartland virus may have a nucleotide sequence of SEQ ID NO: 6 (or a nucleotide sequence having at least 80%, 85%, 90%, 95%, 96%, 97%, 98%, 99% or 99% identity to SEQ ID NO: 6). In another embodiment, the ORF encoding Gn of Heartland virus fused with COL1A1 has a nucleotide sequence of SEQ ID NO: 7 (or a nucleotide sequence having at least 80%, 85%, 90%, 95%, 96%, 97%, 98%, 99% or 99% identity to SEQ ID NO: 7). In some embodiment, the ORF encoding Gc of Heartland virus fused with COL1A1 has a nucleotide sequence of SEQ ID NO: 8 (or a nucleotide sequence having at least 80%, 85%, 90%, 95%, 96%, 97%, 98%, 99% or 99% identity to SEQ ID NO: 8).

In the Heartland virus vaccine composition (1), the mRNA comprising the ORF encoding Gn of Heartland virus may further comprise a 5′ untranslated region (UTR), a 3′ UTR, and a poly (A) tail so as to have the structure of 5′UTR-ORF encoding Gn of Heartland virus-3′UTR-poly (A) tail, and the ORF encoding Gn of Heartland virus may have a nucleotide sequence of SEQ ID NO: 5 (or a nucleotide sequence having at least 80%, 85%, 90%, 95%, 96%, 97%, 98%, 99% or 99% identity to SEQ ID NO: 5).

In the Heartland virus vaccine composition (2), the mRNA comprising the ORF encoding Gc of Heartland virus may further comprise a 5′ untranslated region (UTR), a 3′ UTR, and a poly (A) tail so as to have the structure of 5′UTR-ORF encoding Gc of Heartland virus-3′UTR-poly (A) tail, and the ORF encoding Gc of Heartland virus may have a nucleotide sequence of SEQ ID NO: 6 (or a nucleotide sequence having at least 80%, 85%, 90%, 95%, 96%, 97%, 98%, 99% or 99% identity to SEQ ID NO: 6).

In the Heartland virus vaccine composition (3), the mRNA comprising the ORF encoding Gn of Heartland virus fused with COL1A1 signal peptide may further comprise a 5′ untranslated region (UTR), a 3′ UTR, and a poly (A) tail so as to have the structure of 5′UTR-ORF encoding Gn of Heartland virus fused with COL1A1 signal peptide-3′UTR-poly (A) tail, and the ORF encoding Gn of Heartland virus may have a nucleotide sequence of SEQ ID NO: 7 (or a nucleotide sequence having at least 80%, 85%, 90%, 95%, 96%, 97%, 98%, 99% or 99% identity to SEQ ID NO: 7).

In the Heartland virus vaccine composition (4), the mRNA comprising the ORF encoding Gc of Heartland virus fused with COL1A1 signal peptide may further comprise a 5′ untranslated region (UTR), a 3′ UTR, and a poly (A) tail so as to have the structure of 5′UTR-ORF encoding Gc of Heartland virus fused with COL1A1 signal peptide-3′UTR-poly (A) tail, and the ORF encoding Gc of Heartland virus may have a nucleotide sequence of SEQ ID NO: 8 (or a nucleotide sequence having at least 80%, 85%, 90%, 95%, 96%, 97%, 98%, 99% or 99% identity to SEQ ID NO: 8).

In the Heartland virus vaccine composition (1), the mRNA having the structure of 5′UTR-ORF encoding Gn of Heartland virus-3′UTR-poly (A) tail may have a nucleotide sequence of SEQ ID NO: 9 (or a nucleotide sequence having at least 80%, 85%, 90%, 95%, 96%, 97%, 98%, 99% or 99% identity to SEQ ID NO: 9).

In the Heartland virus vaccine composition (2), the mRNA having the structure of 5′UTR-ORF encoding Gc of Heartland virus-3′UTR-poly (A) tail may have a nucleotide sequence of SEQ ID NO: 10 (or a nucleotide sequence having at least 80%, 85%, 90%, 95%, 96%, 97%, 98%, 99% or 99% identity to SEQ ID NO: 10).

In the Heartland virus vaccine composition (3), the mRNA having the structure of 5′UTR-ORF encoding Gn of Heartland virus fused with COL1A1-3′UTR-poly (A) tail may have a nucleotide sequence of SEQ ID NO: 11 (or a nucleotide sequence having at least 80%, 85%, 90%, 95%, 96%, 97%, 98%, 99% or 99% identity to SEQ ID NO: 11).

In the Heartland virus vaccine composition (4), the mRNA having the structure of 5′UTR-ORF encoding Gc of Heartland virus fused with COL1A1-3′UTR-poly (A) tail may have a nucleotide sequence of SEQ ID NO: 12 (or a nucleotide sequence having at least 80%, 85%, 90%, 95%, 96%, 97%, 98%, 99% or 99% identity to SEQ ID NO: 12).

In one embodiment, the poly (A) tail has a length of 50-250 nucleotides. In another embodiment, the poly (A) tail has a length of 100-200 nucleotides. In another embodiment, the poly (A) tail has a length of 110-150 nucleotides. In another embodiment, the poly (A) tail has a length of 115-125 nucleotides. In another embodiment, the poly (A) tail has a length of 116-124 nucleotides. In another embodiment, the poly (A) tail has a length of 117-123 nucleotides. In another embodiment, the poly (A) tail has a length of 118-122 nucleotides. In another embodiment, the poly (A) tail has a length of 119-122 nucleotides. In another embodiment, the poly (A) tail has a length of 115 nucleotides. In another embodiment, the poly (A) tail has a length of 116 nucleotides. In another embodiment, the poly (A) tail has a length of 117 nucleotides. In another embodiment, the poly (A) tail has a length of 118 nucleotides. In another embodiment, the poly (A) tail has a length of 119 nucleotides. In another embodiment, the poly (A) tail has a length of 120 nucleotides. In another embodiment, the poly (A) tail has a length of 121 nucleotides. In another embodiment, the poly (A) tail has a length of 122 nucleotides. In another embodiment, the poly (A) tail has a length of 123 nucleotides. In another embodiment, the poly (A) tail has a length of 124 nucleotides. In another embodiment, the poly (A) tail has a length of 125 nucleotides.

In one embodiment, the mRNA of the present disclosure may comprise at least one chemical modification selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In another embodiment, the chemical modification is in the 5-position of the uracil. In another embodiment, the chemical modification is a N1-methylpseudouridine. In another embodiments, the chemical modification is a N1-ethylpseudouridine.

In one embodiment, the Heartland virus vaccine composition further comprises a pharmaceutically acceptable carrier. In another embodiment, the pharmaceutically acceptable carrier may include any substance or vehicle suitable for delivering a mRNA vaccine to a suitable in vivo or ex vivo site. Such a carrier can include, but is not limited to, an adjuvant, an excipient, a lipid particle, etc. The lipid nanoparticle may be a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In one embodiment, the lipid nanoparticle comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), (ii) a neutral lipid selected from DSPC, DPPC, POPC, DOPE and SM, (iii) a sterol, e.g., cholesterol, and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the lipid nanoparticle includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the lipid nanoparticle includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), and PEG-cDMA.

In one embodiment, the lipid nanoparticle includes 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the lipid nanoparticle include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the lipid nanoparticle includes 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the lipid nanoparticle includes about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the lipid nanoparticle includes about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the Heartland virus vaccine composition of the present disclosure may be delivered, localized and/or concentrated in a specific location using the delivery methods described as follows. As a non-limiting example, a subject may be administered an empty polymeric particle prior to, simultaneously with or after delivering the Heartland virus vaccine composition of the present disclosure to the subject. The empty polymeric particle undergoes a change in volume once in contact with the subject and becomes lodged, embedded, immobilized or entrapped at a specific location in the subject.

In another embodiment, the Heartland virus vaccine composition of the present disclosure may be formulated in an active substance release system. For instance, the active substance release system may comprise at least one nanoparticle bonded to an oligonucleotide inhibitor strand which is hybridized with a catalytically active nucleic acid and a compound bonded to at least one substrate molecule bonded to a therapeutically active substance (e.g., polynucleotides described herein), where the therapeutically active substance is released by the cleavage of the substrate molecule by the catalytically active nucleic acid.

In another embodiment, the Heartland virus vaccine composition of the present disclosure may be formulated in a nanoparticle comprising an inner core comprising a non-cellular material and an outer surface comprising a cellular membrane. The cellular membrane may be derived from a cell or a membrane derived from a virus.

In another embodiment, the Heartland virus vaccine composition of the present disclosure may be formulated in porous nanoparticle-supported lipid bilayers (protocells).

In another embodiment, the Heartland virus vaccine composition of the present disclosure may be formulated in polymeric nanoparticles which have a high glass transition temperature.

In another embodiment, the Heartland virus vaccine composition of the present disclosure may be formulated in nanoparticles used in imaging. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component.

The nanoparticles of the present disclosure may further include nutrients such as, but not limited to, those which deficiencies can lead to health hazards from anemia to neural tube defects. As a non-limiting example, the nutrient may be iron in the form of ferrous, ferric salts or elemental iron, iodine, folic acid, vitamins or micronutrients.

In another embodiment, the Heartland virus vaccine composition of the present disclosure may be formulated in a swellable nanoparticle.

In another embodiment, the Heartland virus vaccine composition of the present disclosure may be formulated in polyanhydride nanoparticles.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery. Other physical features the geometrically engineering particles may have include, but are not limited to, fenestrations, angled arms, asymmetry and surface roughness, charge which can alter the interactions with cells and tissues.

In another embodiment, the nanoparticles of the present disclosure may be water soluble nanoparticles. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure are stealth nanoparticles or target-specific stealth nanoparticles. In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle of the present disclosure may be a nanoparticle-nucleic acid hybrid structure having a high density nucleic acid layer. The nanoparticle of the present disclosure may comprise a nucleic acid such as, but not limited to, polynucleotides described herein and/or known in the art.

In one embodiment, at least one of the nanoparticles of the present disclosure may be embedded in in the core a nanostructure or coated with a low density porous 3-D structure or coating which is capable of carrying or associating with at least one payload within or on the surface of the nanostructure.

In one embodiment, the pharmaceutically acceptable carrier is a lipid nanoparticle encapsulating the mRNAs of the present disclosure therein. In another embodiment, the lipid nanoparticle comprises a first lipid nanoparticle encapsulating the mRNA encoding Gn of Heartland virus, a second lipid nanoparticle encapsulating the mRNA encoding Gc of Heartland virus, a third lipid nanoparticle encapsulating the mRNA encoding Gn of Heartland virus fused with COL1A1 signal peptide, and a fourth lipid nanoparticle encapsulating the mRNA encoding Gc of Heartland virus fused with COL1A1 signal peptide therein.

### 2. The Method of Inducing Immune Response Against Heartland Virus

The present disclosure also provides a method of inducing immune response against Heartland virus comprising administering an effective amount of the Heartland virus vaccine composition of the present disclosure a subject in need thereof. In one embodiment, the effective amount of the Heartland virus vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the Heartland virus vaccine (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

Administration of an effective amount (immunogenically effective amount) of the Heartland virus vaccine compositions (e.g., Heartland virus vaccine compositions (1) to (4)) is typically intramuscular or subcutaneous. Thus, the Heartland virus vaccine composition is typically formulated for intramuscular or subcutaneous injection, and for the purposes of the invention formulated without adjuvants, preferably without any adjuvant. However other modes of administration, such as intravenous, cutaneous, intradermal or nasal can be envisaged as well. For intravenous, cutaneous or subcutaneous injection, the adenovirus vector will be in the form of a parenterally acceptable aqueous solution which is pyrogen-free and has suitable pH, isotonicity and stability. Likewise, the isolated envelope polypeptide will be in the form of a parenterally acceptable solution having a suitable pH, isotonicity, and stability. Those of ordinary skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In a particular embodiment, an effective amount (immunogenically effective amount) of the Heartland virus vaccine composition (e.g., Heartland virus vaccine compositions (1) to (4)) is administered via intramuscular administration. Intramuscular administration can be achieved by using a needle to inject a suspension of the adenovirus vectors and/or envelope polypeptides. An alternative is the use of a needleless injection device to administer the composition (using, e.g., Biojector™) or a freeze-dried powder containing the vaccine.

In one embodiment, the priming immunization and/or the boosting administration, preferably both the priming and boosting administration, further comprise administering one or more adenovirus vectors that encode one or more further Heartland virus antigens.

The timing for administering priming and boosting immunizations is not particularly limited. For example, a vaccine composition can be administered for priming immunization, and re-administered prior to administration of a vaccine composition for boosting immunization. Further administrations of a vaccine composition for further boosting immunizations are also contemplated. In certain embodiments, a booster vaccine is first administered about 1-12 weeks, e.g., 1, 2, 3, 4, 5, 6, 7, 8, 9, 10, 11, or 12 weeks after a primer vaccine is initially administered. In other embodiments, a booster vaccine is first administered about 12-52 weeks, e.g., about 12, 14, 16, 18, 20, 22, 24, 26, 28, 30, 32, 34, 36, 38, 40, 42, 44, 46, 48, 50, or 52 weeks after a primer vaccine is initially administered. One of ordinary skill in the art will be able to vary the exact timing of the priming and boosting vaccines, frequency of administration thereof, dosage thereof, etc., based upon the teachings herein and general knowledge in the art.

In one embodiment, the Heartland virus vaccine composition may comprise mRNA comprising the ORF encoding Gn of heartland virus, mRNA comprising the ORF encoding Gc of heartland virus, mRNA comprising the ORF encoding Gn of heartland virus fused with COL1A1 signal peptide, and mRNA comprising the ORF encoding Gc of heartland virus fused with COL1A1 signal peptide, formulated in a lipid nanoparticle comprising MC3, Cholesterol, DSPC and PEG2000-DMG, the buffer trisodium citrate, sucrose and water for injection. As a non-limiting example, the composition may comprise 2.0 mg/mL of drug substance (e.g., Heartland virus vaccine compositions (1) to (4)), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In one embodiment, a method of inducing immune response against Heartland virus comprises administering an effective amount of the Heartland virus vaccine composition (1) of the present disclosure to a subject in need thereof. In the Heartland virus vaccine composition (1), the mRNA having the structure of 5′UTR-ORF encoding Gn of Heartland virus-3′UTR-poly (A) tail may have a nucleotide sequence of SEQ ID NO: 9 (or a nucleotide sequence having at least 80%, 85%, 90%, 95%, 96%, 97%, 98%, 99% or 99% identity to SEQ ID NO: 9).

In another embodiment, a method of inducing immune response against Heartland virus comprises administering an effective amount of the Heartland virus vaccine composition (2) of the present disclosure to a subject in need thereof. In the Heartland virus vaccine composition (2), the mRNA having the structure of 5′UTR-ORF encoding Gc of Heartland virus-3′UTR-poly (A) tail may have a nucleotide sequence of SEQ ID NO: 10 (or a nucleotide sequence having at least 80%, 85%, 90%, 95%, 96%, 97%, 98%, 99% or 99% identity to SEQ ID NO: 10).

In another embodiment, a method of inducing immune response against Heartland virus comprises administering an effective amount of the Heartland virus vaccine composition (3) of the present disclosure to a subject in need thereof. In the Heartland virus vaccine composition (3), the mRNA having the structure of 5′UTR-ORF encoding Gn of Heartland virus fused with COL1A1-3′UTR-poly (A) tail may have a nucleotide sequence of SEQ ID NO: 11 (or a nucleotide sequence having at least 80%, 85%, 90%, 95%, 96%, 97%, 98%, 99% or 99% identity to SEQ ID NO: 11).

In another embodiment, a method of inducing immune response against Heartland virus comprises administering an effective amount of the Heartland virus vaccine composition (4) of the present disclosure to a subject in need thereof. In the Heartland virus vaccine composition (4), the mRNA having the structure of 5′UTR-ORF encoding Gc of Heartland virus fused with COL1A1-3′UTR-poly (A) tail may have a nucleotide sequence of SEQ ID NO: 12 (or a nucleotide sequence having at least 80%, 85%, 90%, 95%, 96%, 97%, 98%, 99% or 99% identity to SEQ ID NO: 12).

### 3. Sequence Information

The specific sequence information of SEQ ID NOS: 1 to 8 cited in the present disclosure is as follows.

1) Protein sequence of the ORF encoded in Gn of heartland virus
SEQ ID NO: 1
MIVPIVLFLTLCPSELSAWGSPGDPIVCGVRTETNKSIQIEWKEGRSEKLCQIDRLGHVTS
WLRNHSSFQGLIGQVKGRPSVSYFPEGASYPRWSGLLSPCDAEWLGLIAVSKAGDTDMI
VPGPTYKGKIFVERPTYNGYKGWGCADGKSLSHSGTYCETDSSVSSGLIQGDRVLWVG
EVVCQRGTPVPEDVFSELVSLSQSEFPDVCKIDGVALNQCEQESIPQPLDVAWIDVGRSH
KVLMREHKTKWVQESSAKDFVCFKVGQGPCSKQEEDDCMSKGNCHGDEVFCRMAGC
SARMQDNQEGCRCELLQKPGEIIVNYGGVSVRPTCYGFSRMMATLEVHKPDRELTGCT
GCHLECIEGGVKIVTLTSELRSATVCASHFCASAKGGSKTTDILFHTGALVGPNSIRITGQ
LLDGSKFSFDGHCIFPDGCMALDCTFCKEFLRNPQCYPVKKWLFLVVVVMCCYCALML
LTNILRAIGVWGTWVFAPIKLALALGLRLAKLSKKGLVAVVTRGQMIVNDELHQIRVER
GEQNEGRQG
2) Protein sequence of the ORF encoded in Gc of heartland virus
SEQ ID NO: 2
MYGPRGPIRHWLYSPALILILTTSICSGCDELVHAESKSITCKSASGNEKECSVTGRALLP
AVNPGQEACLHFSMPGSPDSKCLKIKVKSINLRCKQASSYYVPEAKARCTSVRRCRWA
GDCQSGCPTYFSSNSFSDDWANRMDRAGLGMSGCSDGCGGAACGCFNAAPSCIFWRK
WVENPSNRVWKVSPCASWVLAATIELTLPSGEVKTLEPVTGQATQMFKGVAITYLGSSI
EIVGMTRLCEMKEMGTGIMALAPCNDPGHAIMGNVGEIQCSSIESAKHIRSDGCIWNAD
LVGIELRVDDAVCFSKLTSVEAVANFSKIPATISGVRFDQGNHGESRIYGSPLDITRVSGE
FSVSFRGMRLRLSEISASCTGEITNVSGCYSCMTGASVSIKLHSSKNTTGHLKCDSDETAF
SVMEGTHTYRPHMSFDKAVIDEECVLNCGGHSSKLLLKGSLVFMDVPRFVDGSYVQTY
HSKVPAGGRVPNPVDWLNALFGDGITRWILGIIGVLLACVMLFVVVVAITRRLIKGLTQ
RAKVA
3) Protein sequence of the ORF encoded in Gn of heartland virus fused with COL1A1 signal
peptide sequence (COL1A1 signal peptide sequence is underlined)
SEQ ID NO: 3
MFSFVDLRLLLLLAATALLTHGSPGDPIVCGVRTETNKSIQIEWKEGRSEKLCQIDRLGH
VTSWLRNHSSFQGLIGQVKGRPSVSYFPEGASYPRWSGLLSPCDAEWLGLIAVSKAGDT
DMIVPGPTYKGKIFVERPTYNGYKGWGCADGKSLSHSGTYCETDSSVSSGLIQGDRVL
WVGEVVCQRGTPVPEDVFSELVSLSQSEFPDVCKIDGVALNQCEQESIPQPLDVAWIDV
GRSHKVLMREHKTKWVQESSAKDFVCFKVGQGPCSKQEEDDCMSKGNCHGDEVFCR
MAGCSARMQDNQEGCRCELLQKPGEIIVNYGGVSVRPTCYGFSRMMATLEVHKPDREL
TGCTGCHLECIEGGVKIVTLTSELRSATVCASHFCASAKGGSKTTDILFHTGALVGPNSIR
ITGQLLDGSKFSFDGHCIFPDGCMALDCTFCKEFLRNPQCYPVKKWLFLVVVVMCCYC
ALMLLTNILRAIGVWGTWVFAPIKLALALGLRLAKLSKKGLVAVVTRGQMIVNDELHQI
RVERGEQNEGRQG
4) Protein sequence of the ORF encoded in Gc of heartland virus fused with COL1A1 signal
peptide sequence (COL1A1 signal peptide sequence is underlined)
SEQ ID NO: 4
MFSFVDLRLLLLLAATALLTHGCDELVHAESKSITCKSASGNEKECSVTGRALLPAVNP
GQEACLHFSMPGSPDSKCLKIKVKSINLRCKQASSYYVPEAKARCTSVRRCRWAGDCQS
GCPTYFSSNSFSDDWANRMDRAGLGMSGCSDGCGGAACGCFNAAPSCIFWRKWVENP
SNRVWKVSPCASWVLAATIELTLPSGEVKTLEPVTGQATQMFKGVAITYLGSSIEIVGMT
RLCEMKEMGTGIMALAPCNDPGHAIMGNVGEIQCSSIESAKHIRSDGCIWNADLVGIELR
VDDAVCFSKLTSVEAVANFSKIPATISGVRFDQGNHGESRIYGSPLDITRVSGEFSVSFRG
MRLRLSEISASCTGEITNVSGCYSCMTGASVSIKLHSSKNTTGHLKCDSDETAFSVMEGT
HTYRPHMSFDKAVIDEECVLNCGGHSSKLLLKGSLVFMDVPRFVDGSYVQTYHSKVPA
GGRVPNPVDWLNALFGDGITRWILGIIGVLLACVMLFVVVVAITRRLIKGLTQRAKVA
5) Gn of heartland virus mRNA sequence (ORF)
SEQ ID NO: 5
AUGAUCGUGCCCAUUGUCCUGUUUCUCACGCUCUGUCCGUCCGAACUCAGUGCCU
GGGGCUCUCCAGGAGACCCUAUUGUUUGUGGUGUGAGGACUGAAACAAACAAAU
CCAUUCAGAUUGAGUGGAAGGAGGGGAGAUCAGAGAAGCUGUGCCAGAUUGACA
GGCUUGGACAUGUCACAAGCUGGUUAAGAAACCACUCAUCUUUCCAGGGGCUUA
UUGGUCAGGUGAAGGGAAGACCAAGUGUUUCCUACUUCCCAGAAGGGGCUUCUU
ACCCAAGGUGGAGCGGCCUAUUGAGCCCAUGUGAUGCUGAAUGGCUGGGACUGA
UAGCAGUGAGCAAGGCUGGAGACACAGACAUGAUUGUCCCAGGCCCAACUUACAA
AGGCAAAAUCUUUGUUGAGAGACCAACAUACAACGGUUACAAAGGCUGGGGGUG
UGCAGAUGGAAAGUCACUAAGCCACUCAGGCACAUAUUGUGAAACUGACAGCUC
AGUGAGUUCUGGUUUAAUUCAGGGAGAUAGGGUUCUCUGGGUUGGGGAAGUGGU
CUGUCAGAGAGGGACCCCUGUGCCAGAAGAUGUAUUUAGUGAACUGGUUAGCUU
GAGUCAAAGUGAGUUCCCAGAUGUGUGCAAGAUUGAUGGUGUUGCAUUGAACCA
GUGUGAGCAGGAGAGCAUCCCCCAGCCACUGGACGUUGCAUGGAUUGAUGUUGG
AAGGUCUCAUAAGGUACUGAUGAGAGAACACAAAACUAAAUGGGUCCAAGAGAG
CUCAGCAAAGGACUUUGUGUGCUUCAAGGUGGGUCAGGGGCCGUGUUCAAAACA
AGAGGAAGAUGACUGCAUGAGUAAGGGCAACUGCCAUGGGGAUGAGGUUUUCUG
UAGGAUGGCAGGAUGCUCUGCCCGCAUGCAAGAUAAUCAAGAAGGCUGUAGGUG
CGAACUGCUUCAAAAACCUGGAGAAAUCAUUGUGAAUUAUGGAGGCGUCUCUGU
GAGACCAACCUGUUAUGGAUUCUCCAGAAUGAUGGCAACAUUGGAAGUUCACAA
ACCUGAUAGAGAAUUAACAGGGUGCACGGGUUGUCACCUAGAGUGCAUAGAGGG
AGGAGUUAAAAUUGUAACGCUUACAAGCGAGCUGAGAAGUGCAACAGUCUGUGC
UUCACACUUUUGUGCAUCUGCAAAGGGGGGCUCAAAGACAACUGACAUACUCUUC
CACACUGGUGCUCUCGUUGGACCCAAUUCCAUUAGAAUAACUGGCCAGUUGUUAG
AUGGGAGCAAGUUUUCCUUUGAUGGGCACUGCAUAUUCCCAGAUGGGUGCAUGG
CACUUGACUGCACCUUCUGUAAGGAGUUCCUGAGAAACCCACAAUGUUACCCAGU
GAAGAAAUGGCUGUUCCUGGUGGUAGUUGUAAUGUGCUGCUAUUGCGCCCUGAU
GCUGCUUACUAACAUACUGAGAGCUAUAGGUGUUUGGGGGACAUGGGUUUUUGC
UCCAAUAAAGUUGGCUCUAGCAUUAGGGUUGAGGCUUGCCAAACUGUCAAAGAA
GGGGUUGGUUGCUGUGGUUACAAGGGGCCAAAUGAUCGUGAAUGAUGAGCUGCA
CCAGAUUCGAGUGGAGAGAGGUGAGCAAAAUGAGGGAAGACAAGGU
6) Gc of heartland virus mRNA sequence (ORF)
SEQ ID NO: 6
AUGUACGGACCUAGAGGCCCCAUUCGUCACUGGCUAUACUCACCUGCCCUUAUUC
UCAUUCUCACCACUUCAAUUUGCUCUGGAUGUGAUGAGCUUGUUCAUGCUGAGA
GUAAAUCUAUCACAUGCAAGUCUGCAUCUGGGAAUGAGAAGGAGUGCUCAGUGA
CAGGCAGAGCUUUGCUCCCAGCUGUUAAUCCAGGGCAGGAGGCCUGCUUGCACUU
CAGCAUGCCAGGAAGCCCAGACUCUAAGUGCCUCAAGAUCAAAGUGAAAUCAAUA
AAUCUCAGGUGUAAGCAAGCCUCUUCAUAUUAUGUUCCUGAAGCAAAGGCAAGA
UGUACAUCUGUCAGAAGGUGCAGGUGGGCAGGUGACUGUCAAUCUGGGUGUCCA
ACAUAUUUCAGCUCGAACUCAUUCUCAGAUGAUUGGGCAAACAGGAUGGACAGG
GCUGGGCUCGGGAUGAGUGGGUGCUCAGAUGGGUGUGGUGGAGCUGCAUGUGGG
UGUUUCAAUGCAGCGCCAUCCUGCAUCUUUUGGAGAAAGUGGGUGGAGAACCCA
UCCAAUCGUGUCUGGAAGGUGUCACCUUGUGCAUCAUGGGUGCUAGCUGCAACCA
UUGAGUUGACCCUGCCAUCAGGAGAGGUUAAGACUCUAGAGCCUGUCACAGGGC
AAGCAACUCAGAUGUUCAAGGGUGUUGCAAUCACAUAUCUGGGAUCAUCCAUUG
AGAUUGUUGGCAUGACCAGGCUAUGUGAGAUGAAAGAGAUGGGAACUGGGAUAA
UGGCACUAGCCCCCUGCAAUGAUCCAGGGCACGCCAUAAUGGGAAAUGUGGGUGA
GAUCCAAUGCAGUAGUAUAGAAAGCGCAAAGCACAUCAGAUCUGAUGGGUGCAU
UUGGAAUGCUGACCUAGUUGGGAUAGAAUUGAGGGUUGAUGAUGCUGUGUGUUU
CUCGAAACUCACUAGUGUUGAGGCAGUUGCAAAUUUUUCAAAAAUCCCGGCAAC
AAUUUCUGGGGUUCGCUUUGAUCAAGGGAAUCAUGGAGAAUCACGUAUCUAUGG
UAGCCCAUUAGAUAUCACGAGGGUUAGUGGGGAAUUCUCAGUGUCAUUCAGAGG
GAUGAGGCUCAGACUAUCUGAGAUAUCAGCAAGCUGCACAGGUGAGAUAACAAA
CGUCUCUGGUUGUUACUCCUGCAUGACCGGGGCCUCAGUCAGCAUAAAGUUGCAU
AGCAGUAAGAACACAACAGGUCAUCUUAAGUGUGAUUCAGAUGAGACCGCAUUC
AGUGUCAUGGAGGGAACACACACAUAUAGGCCUCACAUGAGCUUUGAUAAAGCA
GUAAUAGAUGAGGAGUGUGUGCUAAACUGUGGUGGCCACUCAUCAAAACUGCUG
CUCAAAGGGAGCCUUGUUUUCAUGGACGUGCCAAGGUUUGUUGAUGGGAGUUAU
GUCCAAACAUAUCACAGCAAGGUGCCUGCUGGGGGAAGGGUCCCAAAUCCAGUAG
ACUGGCUCAACGCACUGUUUGGAGAUGGCAUAACACGAUGGAUUCUUGGGAUUA
UAGGGGUUCUGCUGGCAUGUGUCAUGCUAUUUGUGGUGGUGGUUGCCAUCACUA
GGCGAUUGAUCAAGGGACUGACUCAAAGGGCGAAGGUGGCA
7) Gn of heartland virus fused with COL1A1 signal peptide mRNA sequence (ORF)
SEQ ID NO: 7
AUGUUCAGCUUCGUGGACCUGAGACUGCUGCUGCUACUGGCCGCUACAGCCCUGC
UGACCCACGGCUCUCCAGGAGACCCUAUUGUUUGUGGUGUGAGGACUGAAACAA
ACAAAUCCAUUCAGAUUGAGUGGAAGGAGGGGAGAUCAGAGAAGCUGUGCCAGA
UUGACAGGCUUGGACAUGUCACAAGCUGGUUAAGAAACCACUCAUCUUUCCAGG
GGCUUAUUGGUCAGGUGAAGGGAAGACCAAGUGUUUCCUACUUCCCAGAAGGGG
CUUCUUACCCAAGGUGGAGCGGCCUAUUGAGCCCAUGUGAUGCUGAAUGGCUGG
GACUGAUAGCAGUGAGCAAGGCUGGAGACACAGACAUGAUUGUCCCAGGCCCAAC
UUACAAAGGCAAAAUCUUUGUUGAGAGACCAACAUACAACGGUUACAAAGGCUG
GGGGUGUGCAGAUGGAAAGUCACUAAGCCACUCAGGCACAUAUUGUGAAACUGA
CAGCUCAGUGAGUUCUGGUUUAAUUCAGGGAGAUAGGGUUCUCUGGGUUGGGGA
AGUGGUCUGUCAGAGAGGGACCCCUGUGCCAGAAGAUGUAUUUAGUGAACUGGU
UAGCUUGAGUCAAAGUGAGUUCCCAGAUGUGUGCAAGAUUGAUGGUGUUGCAUU
GAACCAGUGUGAGCAGGAGAGCAUCCCCCAGCCACUGGACGUUGCAUGGAUUGAU
GUUGGAAGGUCUCAUAAGGUACUGAUGAGAGAACACAAAACUAAAUGGGUCCAA
GAGAGCUCAGCAAAGGACUUUGUGUGCUUCAAGGUGGGUCAGGGGCCGUGUUCA
AAACAAGAGGAAGAUGACUGCAUGAGUAAGGGCAACUGCCAUGGGGAUGAGGUU
UUCUGUAGGAUGGCAGGAUGCUCUGCCCGCAUGCAAGAUAAUCAAGAAGGCUGU
AGGUGCGAACUGCUUCAAAAACCUGGAGAAAUCAUUGUGAAUUAUGGAGGCGUC
UCUGUGAGACCAACCUGUUAUGGAUUCUCCAGAAUGAUGGCAACAUUGGAAGUU
CACAAACCUGAUAGAGAAUUAACAGGGUGCACGGGUUGUCACCUAGAGUGCAUA
GAGGGAGGAGUUAAAAUUGUAACGCUUACAAGCGAGCUGAGAAGUGCAACAGUC
UGUGCUUCACACUUUUGUGCAUCUGCAAAGGGGGGCUCAAAGACAACUGACAUA
CUCUUCCACACUGGUGCUCUCGUUGGACCCAAUUCCAUUAGAAUAACUGGCCAGU
UGUUAGAUGGGAGCAAGUUUUCCUUUGAUGGGCACUGCAUAUUCCCAGAUGGGU
GCAUGGCACUUGACUGCACCUUCUGUAAGGAGUUCCUGAGAAACCCACAAUGUUA
CCCAGUGAAGAAAUGGCUGUUCCUGGUGGUAGUUGUAAUGUGCUGCUAUUGCGC
CCUGAUGCUGCUUACUAACAUACUGAGAGCUAUAGGUGUUUGGGGGACAUGGGU
UUUUGCUCCAAUAAAGUUGGCUCUAGCAUUAGGGUUGAGGCUUGCCAAACUGUC
AAAGAAGGGGUUGGUUGCUGUGGUUACAAGGGGCCAAAUGAUCGUGAAUGAUGA
GCUGCACCAGAUUCGAGUGGAGAGAGGUGAGCAAAAUGAGGGAAGACAAGGU
8) Gc of heartland virus fused with COL1A1 signal peptide mRNA sequence (ORF)
SEQ ID NO: 8
AUGUUCAGCUUCGUGGACCUGAGACUGCUGCUGCUACUGGCCGCUACAGCCCUGC
UGACCCACGGCUGUGAUGAGCUUGUUCAUGCUGAGAGUAAAUCUAUCACAUGCA
AGUCUGCAUCUGGGAAUGAGAAGGAGUGCUCAGUGACAGGCAGAGCUUUGCUCC
CAGCUGUUAAUCCAGGGCAGGAGGCCUGCUUGCACUUCAGCAUGCCAGGAAGCCC
AGACUCUAAGUGCCUCAAGAUCAAAGUGAAAUCAAUAAAUCUCAGGUGUAAGCA
AGCCUCUUCAUAUUAUGUUCCUGAAGCAAAGGCAAGAUGUACAUCUGUCAGAAG
GUGCAGGUGGGCAGGUGACUGUCAAUCUGGGUGUCCAACAUAUUUCAGCUCGAA
CUCAUUCUCAGAUGAUUGGGCAAACAGGAUGGACAGGGCUGGGCUCGGGAUGAG
UGGGUGCUCAGAUGGGUGUGGUGGAGCUGCAUGUGGGUGUUUCAAUGCAGCGCC
AUCCUGCAUCUUUUGGAGAAAGUGGGUGGAGAACCCAUCCAAUCGUGUCUGGAA
GGUGUCACCUUGUGCAUCAUGGGUGCUAGCUGCAACCAUUGAGUUGACCCUGCCA
UCAGGAGAGGUUAAGACUCUAGAGCCUGUCACAGGGCAAGCAACUCAGAUGUUC
AAGGGUGUUGCAAUCACAUAUCUGGGAUCAUCCAUUGAGAUUGUUGGCAUGACC
AGGCUAUGUGAGAUGAAAGAGAUGGGAACUGGGAUAAUGGCACUAGCCCCCUGC
AAUGAUCCAGGGCACGCCAUAAUGGGAAAUGUGGGUGAGAUCCAAUGCAGUAGU
AUAGAAAGCGCAAAGCACAUCAGAUCUGAUGGGUGCAUUUGGAAUGCUGACCUA
GUUGGGAUAGAAUUGAGGGUUGAUGAUGCUGUGUGUUUCUCGAAACUCACUAGU
GUUGAGGCAGUUGCAAAUUUUUCAAAAAUCCCGGCAACAAUUUCUGGGGUUCGC
UUUGAUCAAGGGAAUCAUGGAGAAUCACGUAUCUAUGGUAGCCCAUUAGAUAUC
ACGAGGGUUAGUGGGGAAUUCUCAGUGUCAUUCAGAGGGAUGAGGCUCAGACUA
UCUGAGAUAUCAGCAAGCUGCACAGGUGAGAUAACAAACGUCUCUGGUUGUUAC
UCCUGCAUGACCGGGGCCUCAGUCAGCAUAAAGUUGCAUAGCAGUAAGAACACAA
CAGGUCAUCUUAAGUGUGAUUCAGAUGAGACCGCAUUCAGUGUCAUGGAGGGAA
CACACACAUAUAGGCCUCACAUGAGCUUUGAUAAAGCAGUAAUAGAUGAGGAGU
GUGUGCUAAACUGUGGUGGCCACUCAUCAAAACUGCUGCUCAAAGGGAGCCUUG
UUUUCAUGGACGUGCCAAGGUUUGUUGAUGGGAGUUAUGUCCAAACAUAUCACA
GCAAGGUGCCUGCUGGGGGAAGGGUCCCAAAUCCAGUAGACUGGCUCAACGCACU
GUUUGGAGAUGGCAUAACACGAUGGAUUCUUGGGAUUAUAGGGGUUCUGCUGGC
AUGUGUCAUGCUAUUUGUGGUGGUGGUUGCCAUCACUAGGCGAUUGAUCAAGGG
ACUGACUCAAAGGGCGAAGGUGGCA
9) Gn of heartland virus mRNA sequence (5′UTR-ORF-3′UTR-poly (A) tail)
SEQ ID NO: 9
AGGCCGGCACUCUUCUGGUCCCCACAGACUCAGAGAGAACCCGCCGCCACCAUGA
UCGUGCCCAUUGUCCUGUUUCUCACGCUCUGUCCGUCCGAACUCAGUGCCUGGGG
CUCUCCAGGAGACCCUAUUGUUUGUGGUGUGAGGACUGAAACAAACAAAUCCAU
UCAGAUUGAGUGGAAGGAGGGGAGAUCAGAGAAGCUGUGCCAGAUUGACAGGCU
UGGACAUGUCACAAGCUGGUUAAGAAACCACUCAUCUUUCCAGGGGCUUAUUGG
UCAGGUGAAGGGAAGACCAAGUGUUUCCUACUUCCCAGAAGGGGCUUCUUACCCA
AGGUGGAGCGGCCUAUUGAGCCCAUGUGAUGCUGAAUGGCUGGGACUGAUAGCA
GUGAGCAAGGCUGGAGACACAGACAUGAUUGUCCCAGGCCCAACUUACAAAGGCA
AAAUCUUUGUUGAGAGACCAACAUACAACGGUUACAAAGGCUGGGGGUGUGCAG
AUGGAAAGUCACUAAGCCACUCAGGCACAUAUUGUGAAACUGACAGCUCAGUGA
GUUCUGGUUUAAUUCAGGGAGAUAGGGUUCUCUGGGUUGGGGAAGUGGUCUGUC
AGAGAGGGACCCCUGUGCCAGAAGAUGUAUUUAGUGAACUGGUUAGCUUGAGUC
AAAGUGAGUUCCCAGAUGUGUGCAAGAUUGAUGGUGUUGCAUUGAACCAGUGUG
AGCAGGAGAGCAUCCCCCAGCCACUGGACGUUGCAUGGAUUGAUGUUGGAAGGU
CUCAUAAGGUACUGAUGAGAGAACACAAAACUAAAUGGGUCCAAGAGAGCUCAG
CAAAGGACUUUGUGUGCUUCAAGGUGGGUCAGGGGCCGUGUUCAAAACAAGAGG
AAGAUGACUGCAUGAGUAAGGGCAACUGCCAUGGGGAUGAGGUUUUCUGUAGGA
UGGCAGGAUGCUCUGCCCGCAUGCAAGAUAAUCAAGAAGGCUGUAGGUGCGAAC
UGCUUCAAAAACCUGGAGAAAUCAUUGUGAAUUAUGGAGGCGUCUCUGUGAGAC
CAACCUGUUAUGGAUUCUCCAGAAUGAUGGCAACAUUGGAAGUUCACAAACCUG
AUAGAGAAUUAACAGGGUGCACGGGUUGUCACCUAGAGUGCAUAGAGGGAGGAG
UUAAAAUUGUAACGCUUACAAGCGAGCUGAGAAGUGCAACAGUCUGUGCUUCAC
ACUUUUGUGCAUCUGCAAAGGGGGGCUCAAAGACAACUGACAUACUCUUCCACAC
UGGUGCUCUCGUUGGACCCAAUUCCAUUAGAAUAACUGGCCAGUUGUUAGAUGG
GAGCAAGUUUUCCUUUGAUGGGCACUGCAUAUUCCCAGAUGGGUGCAUGGCACU
UGACUGCACCUUCUGUAAGGAGUUCCUGAGAAACCCACAAUGUUACCCAGUGAAG
AAAUGGCUGUUCCUGGUGGUAGUUGUAAUGUGCUGCUAUUGCGCCCUGAUGCUG
CUUACUAACAUACUGAGAGCUAUAGGUGUUUGGGGGACAUGGGUUUUUGCUCCA
AUAAAGUUGGCUCUAGCAUUAGGGUUGAGGCUUGCCAAACUGUCAAAGAAGGGG
UUGGUUGCUGUGGUUACAAGGGGCCAAAUGAUCGUGAAUGAUGAGCUGCACCAG
AUUCGAGUGGAGAGAGGUGAGCAAAAUGAGGGAAGACAAGGUUGAUAAAGCUGG
AGCCUCGGUGGCCUUGCUUCUUGCCCCUUGGGCCUCCCCCCAGCCCCUCCUCCCCU
UCCUGCACCCGUACCCCCGUGGUCUUUGAAUAAAGUCUGAGUGGGCGGCAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAA
10) Gc of heartland virus mRNA sequence (5′UTR-ORF-3′UTR-poly (A) tail)
SEQ ID NO: 10
AGGCCGGCACUCUUCUGGUCCCCACAGACUCAGAGAGAACCCGCCGCCACCAUGU
ACGGACCUAGAGGCCCCAUUCGUCACUGGCUAUACUCACCUGCCCUUAUUCUCAU
UCUCACCACUUCAAUUUGCUCUGGAUGUGAUGAGCUUGUUCAUGCUGAGAGUAA
AUCUAUCACAUGCAAGUCUGCAUCUGGGAAUGAGAAGGAGUGCUCAGUGACAGG
CAGAGCUUUGCUCCCAGCUGUUAAUCCAGGGCAGGAGGCCUGCUUGCACUUCAGC
AUGCCAGGAAGCCCAGACUCUAAGUGCCUCAAGAUCAAAGUGAAAUCAAUAAAU
CUCAGGUGUAAGCAAGCCUCUUCAUAUUAUGUUCCUGAAGCAAAGGCAAGAUGU
ACAUCUGUCAGAAGGUGCAGGUGGGCAGGUGACUGUCAAUCUGGGUGUCCAACA
UAUUUCAGCUCGAACUCAUUCUCAGAUGAUUGGGCAAACAGGAUGGACAGGGCU
GGGCUCGGGAUGAGUGGGUGCUCAGAUGGGUGUGGUGGAGCUGCAUGUGGGUGU
UUCAAUGCAGCGCCAUCCUGCAUCUUUUGGAGAAAGUGGGUGGAGAACCCAUCCA
AUCGUGUCUGGAAGGUGUCACCUUGUGCAUCAUGGGUGCUAGCUGCAACCAUUG
AGUUGACCCUGCCAUCAGGAGAGGUUAAGACUCUAGAGCCUGUCACAGGGCAAGC
AACUCAGAUGUUCAAGGGUGUUGCAAUCACAUAUCUGGGAUCAUCCAUUGAGAU
UGUUGGCAUGACCAGGCUAUGUGAGAUGAAAGAGAUGGGAACUGGGAUAAUGGC
ACUAGCCCCCUGCAAUGAUCCAGGGCACGCCAUAAUGGGAAAUGUGGGUGAGAUC
CAAUGCAGUAGUAUAGAAAGCGCAAAGCACAUCAGAUCUGAUGGGUGCAUUUGG
AAUGCUGACCUAGUUGGGAUAGAAUUGAGGGUUGAUGAUGCUGUGUGUUUCUCG
AAACUCACUAGUGUUGAGGCAGUUGCAAAUUUUUCAAAAAUCCCGGCAACAAUU
UCUGGGGUUCGCUUUGAUCAAGGGAAUCAUGGAGAAUCACGUAUCUAUGGUAGC
CCAUUAGAUAUCACGAGGGUUAGUGGGGAAUUCUCAGUGUCAUUCAGAGGGAUG
AGGCUCAGACUAUCUGAGAUAUCAGCAAGCUGCACAGGUGAGAUAACAAACGUC
UCUGGUUGUUACUCCUGCAUGACCGGGGCCUCAGUCAGCAUAAAGUUGCAUAGCA
GUAAGAACACAACAGGUCAUCUUAAGUGUGAUUCAGAUGAGACCGCAUUCAGUG
UCAUGGAGGGAACACACACAUAUAGGCCUCACAUGAGCUUUGAUAAAGCAGUAA
UAGAUGAGGAGUGUGUGCUAAACUGUGGUGGCCACUCAUCAAAACUGCUGCUCA
AAGGGAGCCUUGUUUUCAUGGACGUGCCAAGGUUUGUUGAUGGGAGUUAUGUCC
AAACAUAUCACAGCAAGGUGCCUGCUGGGGGAAGGGUCCCAAAUCCAGUAGACU
GGCUCAACGCACUGUUUGGAGAUGGCAUAACACGAUGGAUUCUUGGGAUUAUAG
GGGUUCUGCUGGCAUGUGUCAUGCUAUUUGUGGUGGUGGUUGCCAUCACUAGGC
GAUUGAUCAAGGGACUGACUCAAAGGGCGAAGGUGGCAUGAUAAAGCUGGAGCC
UCGGUGGCCUUGCUUCUUGCCCCUUGGGCCUCCCCCCAGCCCCUCCUCCCCUUCCU
GCACCCGUACCCCCGUGGUCUUUGAAUAAAGUCUGAGUGGGCGGCAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAA
11) Gn of heartland virus fused with COL1A1 signal peptide mRNA sequence (5′UTR-ORF-
3′UTR-poly (A) tail)
SEQ ID NO: 11
AGGCCGGCACUCUUCUGGUCCCCACAGACUCAGAGAGAACCCGCCGCCACCAUGU
UCAGCUUCGUGGACCUGAGACUGCUGCUGCUACUGGCCGCUACAGCCCUGCUGAC
CCACGGCUCUCCAGGAGACCCUAUUGUUUGUGGUGUGAGGACUGAAACAAACAA
AUCCAUUCAGAUUGAGUGGAAGGAGGGGAGAUCAGAGAAGCUGUGCCAGAUUGA
CAGGCUUGGACAUGUCACAAGCUGGUUAAGAAACCACUCAUCUUUCCAGGGGCUU
AUUGGUCAGGUGAAGGGAAGACCAAGUGUUUCCUACUUCCCAGAAGGGGCUUCU
UACCCAAGGUGGAGCGGCCUAUUGAGCCCAUGUGAUGCUGAAUGGCUGGGACUG
AUAGCAGUGAGCAAGGCUGGAGACACAGACAUGAUUGUCCCAGGCCCAACUUACA
AAGGCAAAAUCUUUGUUGAGAGACCAACAUACAACGGUUACAAAGGCUGGGGGU
GUGCAGAUGGAAAGUCACUAAGCCACUCAGGCACAUAUUGUGAAACUGACAGCU
CAGUGAGUUCUGGUUUAAUUCAGGGAGAUAGGGUUCUCUGGGUUGGGGAAGUGG
UCUGUCAGAGAGGGACCCCUGUGCCAGAAGAUGUAUUUAGUGAACUGGUUAGCU
UGAGUCAAAGUGAGUUCCCAGAUGUGUGCAAGAUUGAUGGUGUUGCAUUGAACC
AGUGUGAGCAGGAGAGCAUCCCCCAGCCACUGGACGUUGCAUGGAUUGAUGUUG
GAAGGUCUCAUAAGGUACUGAUGAGAGAACACAAAACUAAAUGGGUCCAAGAGA
GCUCAGCAAAGGACUUUGUGUGCUUCAAGGUGGGUCAGGGGCCGUGUUCAAAAC
AAGAGGAAGAUGACUGCAUGAGUAAGGGCAACUGCCAUGGGGAUGAGGUUUUCU
GUAGGAUGGCAGGAUGCUCUGCCCGCAUGCAAGAUAAUCAAGAAGGCUGUAGGU
GCGAACUGCUUCAAAAACCUGGAGAAAUCAUUGUGAAUUAUGGAGGCGUCUCUG
UGAGACCAACCUGUUAUGGAUUCUCCAGAAUGAUGGCAACAUUGGAAGUUCACA
AACCUGAUAGAGAAUUAACAGGGUGCACGGGUUGUCACCUAGAGUGCAUAGAGG
GAGGAGUUAAAAUUGUAACGCUUACAAGCGAGCUGAGAAGUGCAACAGUCUGUG
CUUCACACUUUUGUGCAUCUGCAAAGGGGGGCUCAAAGACAACUGACAUACUCUU
CCACACUGGUGCUCUCGUUGGACCCAAUUCCAUUAGAAUAACUGGCCAGUUGUUA
GAUGGGAGCAAGUUUUCCUUUGAUGGGCACUGCAUAUUCCCAGAUGGGUGCAUG
GCACUUGACUGCACCUUCUGUAAGGAGUUCCUGAGAAACCCACAAUGUUACCCAG
UGAAGAAAUGGCUGUUCCUGGUGGUAGUUGUAAUGUGCUGCUAUUGCGCCCUGA
UGCUGCUUACUAACAUACUGAGAGCUAUAGGUGUUUGGGGGACAUGGGUUUUUG
CUCCAAUAAAGUUGGCUCUAGCAUUAGGGUUGAGGCUUGCCAAACUGUCAAAGA
AGGGGUUGGUUGCUGUGGUUACAAGGGGCCAAAUGAUCGUGAAUGAUGAGCUGC
ACCAGAUUCGAGUGGAGAGAGGUGAGCAAAAUGAGGGAAGACAAGGUUGAUAAA
GCUGGAGCCUCGGUGGCCUUGCUUCUUGCCCCUUGGGCCUCCCCCCAGCCCCUCC
UCCCCUUCCUGCACCCGUACCCCCGUGGUCUUUGAAUAAAGUCUGAGUGGGCGGC
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAA
12) Gc of heartland virus fused with COL1A1 signal peptide mRNA sequence (5′UTR-ORF-
3′UTR-poly (A) tail)
SEQ ID NO: 12
AGGCCGGCACUCUUCUGGUCCCCACAGACUCAGAGAGAACCCGCCGCCACCAUGU
UCAGCUUCGUGGACCUGAGACUGCUGCUGCUACUGGCCGCUACAGCCCUGCUGAC
CCACGGCUGUGAUGAGCUUGUUCAUGCUGAGAGUAAAUCUAUCACAUGCAAGUC
UGCAUCUGGGAAUGAGAAGGAGUGCUCAGUGACAGGCAGAGCUUUGCUCCCAGC
UGUUAAUCCAGGGCAGGAGGCCUGCUUGCACUUCAGCAUGCCAGGAAGCCCAGAC
UCUAAGUGCCUCAAGAUCAAAGUGAAAUCAAUAAAUCUCAGGUGUAAGCAAGCC
UCUUCAUAUUAUGUUCCUGAAGCAAAGGCAAGAUGUACAUCUGUCAGAAGGUGC
AGGUGGGCAGGUGACUGUCAAUCUGGGUGUCCAACAUAUUUCAGCUCGAACUCA
UUCUCAGAUGAUUGGGCAAACAGGAUGGACAGGGCUGGGCUCGGGAUGAGUGGG
UGCUCAGAUGGGUGUGGUGGAGCUGCAUGUGGGUGUUUCAAUGCAGCGCCAUCC
UGCAUCUUUUGGAGAAAGUGGGUGGAGAACCCAUCCAAUCGUGUCUGGAAGGUG
UCACCUUGUGCAUCAUGGGUGCUAGCUGCAACCAUUGAGUUGACCCUGCCAUCAG
GAGAGGUUAAGACUCUAGAGCCUGUCACAGGGCAAGCAACUCAGAUGUUCAAGG
GUGUUGCAAUCACAUAUCUGGGAUCAUCCAUUGAGAUUGUUGGCAUGACCAGGC
UAUGUGAGAUGAAAGAGAUGGGAACUGGGAUAAUGGCACUAGCCCCCUGCAAUG
AUCCAGGGCACGCCAUAAUGGGAAAUGUGGGUGAGAUCCAAUGCAGUAGUAUAG
AAAGCGCAAAGCACAUCAGAUCUGAUGGGUGCAUUUGGAAUGCUGACCUAGUUG
GGAUAGAAUUGAGGGUUGAUGAUGCUGUGUGUUUCUCGAAACUCACUAGUGUUG
AGGCAGUUGCAAAUUUUUCAAAAAUCCCGGCAACAAUUUCUGGGGUUCGCUUUG
AUCAAGGGAAUCAUGGAGAAUCACGUAUCUAUGGUAGCCCAUUAGAUAUCACGA
GGGUUAGUGGGGAAUUCUCAGUGUCAUUCAGAGGGAUGAGGCUCAGACUAUCUG
AGAUAUCAGCAAGCUGCACAGGUGAGAUAACAAACGUCUCUGGUUGUUACUCCU
GCAUGACCGGGGCCUCAGUCAGCAUAAAGUUGCAUAGCAGUAAGAACACAACAG
GUCAUCUUAAGUGUGAUUCAGAUGAGACCGCAUUCAGUGUCAUGGAGGGAACAC
ACACAUAUAGGCCUCACAUGAGCUUUGAUAAAGCAGUAAUAGAUGAGGAGUGUG
UGCUAAACUGUGGUGGCCACUCAUCAAAACUGCUGCUCAAAGGGAGCCUUGUUU
UCAUGGACGUGCCAAGGUUUGUUGAUGGGAGUUAUGUCCAAACAUAUCACAGCA
AGGUGCCUGCUGGGGGAAGGGUCCCAAAUCCAGUAGACUGGCUCAACGCACUGUU
UGGAGAUGGCAUAACACGAUGGAUUCUUGGGAUUAUAGGGGUUCUGCUGGCAUG
UGUCAUGCUAUUUGUGGUGGUGGUUGCCAUCACUAGGCGAUUGAUCAAGGGACU
GACUCAAAGGGCGAAGGUGGCAUGAUAAAGCUGGAGCCUCGGUGGCCUUGCUUC
UUGCCCCUUGGGCCUCCCCCCAGCCCCUCCUCCCCUUCCUGCACCCGUACCCCCGU
GGUCUUUGAAUAAAGUCUGAGUGGGCGGCAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAA
13) Sequence of pUC57-Kan plasmid encoding Gn of heartland virus (Gn of heartland virus
mRNA sequence is underlined)
SEQ ID NO: 13
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGATCGT
GCCCATTGTCCTGTTTCTCACGCTCTGTCCGTCCGAACTCAGTGCCTGGGGCTCTCCA
GGAGACCCTATTGTTTGTGGTGTGAGGACTGAAACAAACAAATCCATTCAGATTGAG
TGGAAGGAGGGGAGATCAGAGAAGCTGTGCCAGATTGACAGGCTTGGACATGTCAC
AAGCTGGTTAAGAAACCACTCATCTTTCCAGGGGCTTATTGGTCAGGTGAAGGGAA
GACCAAGTGTTTCCTACTTCCCAGAAGGGGCTTCTTACCCAAGGTGGAGCGGCCTAT
TGAGCCCATGTGATGCTGAATGGCTGGGACTGATAGCAGTGAGCAAGGCTGGAGAC
ACAGACATGATTGTCCCAGGCCCAACTTACAAAGGCAAAATCTTTGTTGAGAGACC
AACATACAACGGTTACAAAGGCTGGGGGTGTGCAGATGGAAAGTCACTAAGCCACT
CAGGCACATATTGTGAAACTGACAGCTCAGTGAGTTCTGGTTTAATTCAGGGAGATA
GGGTTCTCTGGGTTGGGGAAGTGGTCTGTCAGAGAGGGACCCCTGTGCCAGAAGAT
GTATTTAGTGAACTGGTTAGCTTGAGTCAAAGTGAGTTCCCAGATGTGTGCAAGATT
GATGGTGTTGCATTGAACCAGTGTGAGCAGGAGAGCATCCCCCAGCCACTGGACGT
TGCATGGATTGATGTTGGAAGGTCTCATAAGGTACTGATGAGAGAACACAAAACTA
AATGGGTCCAAGAGAGCTCAGCAAAGGACTTTGTGTGCTTCAAGGTGGGTCAGGGG
CCGTGTTCAAAACAAGAGGAAGATGACTGCATGAGTAAGGGCAACTGCCATGGGGA
TGAGGTTTTCTGTAGGATGGCAGGATGCTCTGCCCGCATGCAAGATAATCAAGAAG
GCTGTAGGTGCGAACTGCTTCAAAAACCTGGAGAAATCATTGTGAATTATGGAGGC
GTCTCTGTGAGACCAACCTGTTATGGATTCTCCAGAATGATGGCAACATTGGAAGTT
CACAAACCTGATAGAGAATTAACAGGGTGCACGGGTTGTCACCTAGAGTGCATAGA
GGGAGGAGTTAAAATTGTAACGCTTACAAGCGAGCTGAGAAGTGCAACAGTCTGTG
CTTCACACTTTTGTGCATCTGCAAAGGGGGGCTCAAAGACAACTGACATACTCTTCC
ACACTGGTGCTCTCGTTGGACCCAATTCCATTAGAATAACTGGCCAGTTGTTAGATG
GGAGCAAGTTTTCCTTTGATGGGCACTGCATATTCCCAGATGGGTGCATGGCACTTG
ACTGCACCTTCTGTAAGGAGTTCCTGAGAAACCCACAATGTTACCCAGTGAAGAAAT
GGCTGTTCCTGGTGGTAGTTGTAATGTGCTGCTATTGCGCCCTGATGCTGCTTACTAA
CATACTGAGAGCTATAGGTGTTTGGGGGACATGGGTTTTTGCTCCAATAAAGTTGGC
TCTAGCATTAGGGTTGAGGCTTGCCAAACTGTCAAAGAAGGGGTTGGTTGCTGTGGT
TACAAGGGGCCAAATGATCGTGAATGATGAGCTGCACCAGATTCGAGTGGAGAGAG
GTGAGCAAAATGAGGGAAGACAAGGTTGATAAAGCTGGAGCCTCGGTGGCCTTGCT
TCTTGCCCCTTGGGCCTCCCCCCAGCCCCTCCTCCCCTTCCTGCACCCGTACCCCCGT
GGTCTTTGAATAAAGTCTGAGTGGGCGGCAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAATGAAGAGCATCGGA
TCCCGGGCCCGTCGACTGCAGAGGCCTGCATGCAAGCTTGGTGTAATCATGGTCATA
GCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAAT
GAATCGGCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAGCTCACT
CAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTC
AGTGGAACGAAAACTCACGTTAAGGGATTTTGGTCATGAGATTATCAAAAAGGATC
TTCACCTAGATCCTTTTAAATTAAAAATGAAGTTTTAAATCAAGCCCAATCTGAATA
ATGTTACAACCAATTAACCAATTCTGATTAGAAAAACTCATCGAGCATCAAATGAAA
CTGCAATTTATTCATATCAGGATTATCAATACCATATTTTTGAAAAAGCCGTTTCTGT
AATGAAGGAGAAAACTCACCGAGGCAGTTCCATAGGATGGCAAGATCCTGGTATCG
GTCTGCGATTCCGACTCGTCCAACATCAATACAACCTATTAATTTCCCCTCGTCAAA
AATAAGGTTATCAAGTGAGAAATCACCATGAGTGACGACTGAATCCGGTGAGAATG
GCAAAAGTTTATGCATTTCTTTCCAGACTTGTTCAACAGGCCAGCCATTACGCTCGTC
ATCAAAATCACTCGCATCAACCAAACCGTTATTCATTCGTGATTGCGCCTGAGCGAG
ACGAAATACGCGATCGCTGTTAAAAGGACAATTACAAACAGGAATCGAATGCAACC
GGCGCAGGAACACTGCCAGCGCATCAACAATATTTTCACCTGAATCAGGATATTCTT
CTAATACCTGGAATGCTGTTTTTCCGGGGATCGCAGTGGTGAGTAACCATGCATCAT
CAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCA
GAAACAACTCTGGCGCATCGGGCTTCCCATACAAGCGATAGATTGTCGCACCTGATT
GCCCGACATTATCGCGAGCCCATTTATACCCATATAAATCAGCATCCATGTTGGAAT
TTAATCGCGGCCTCGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACGGGCCAGAGCTGCA
14) Sequence of pUC57-Kan plasmid encoding Gc of heartland virus (Gc of heartland virus
mRNA sequence is underlined)
SEQ ID NO: 14
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGTACG
GACCTAGAGGCCCCATTCGTCACTGGCTATACTCACCTGCCCTTATTCTCATTCTCAC
CACTTCAATTTGCTCTGGATGTGATGAGCTTGTTCATGCTGAGAGTAAATCTATCAC
ATGCAAGTCTGCATCTGGGAATGAGAAGGAGTGCTCAGTGACAGGCAGAGCTTTGC
TCCCAGCTGTTAATCCAGGGCAGGAGGCCTGCTTGCACTTCAGCATGCCAGGAAGCC
CAGACTCTAAGTGCCTCAAGATCAAAGTGAAATCAATAAATCTCAGGTGTAAGCAA
GCCTCTTCATATTATGTTCCTGAAGCAAAGGCAAGATGTACATCTGTCAGAAGGTGC
AGGTGGGCAGGTGACTGTCAATCTGGGTGTCCAACATATTTCAGCTCGAACTCATTC
TCAGATGATTGGGCAAACAGGATGGACAGGGCTGGGCTCGGGATGAGTGGGTGCTC
AGATGGGTGTGGTGGAGCTGCATGTGGGTGTTTCAATGCAGCGCCATCCTGCATCTT
TTGGAGAAAGTGGGTGGAGAACCCATCCAATCGTGTCTGGAAGGTGTCACCTTGTGC
ATCATGGGTGCTAGCTGCAACCATTGAGTTGACCCTGCCATCAGGAGAGGTTAAGAC
TCTAGAGCCTGTCACAGGGCAAGCAACTCAGATGTTCAAGGGTGTTGCAATCACATA
TCTGGGATCATCCATTGAGATTGTTGGCATGACCAGGCTATGTGAGATGAAAGAGAT
GGGAACTGGGATAATGGCACTAGCCCCCTGCAATGATCCAGGGCACGCCATAATGG
GAAATGTGGGTGAGATCCAATGCAGTAGTATAGAAAGCGCAAAGCACATCAGATCT
GATGGGTGCATTTGGAATGCTGACCTAGTTGGGATAGAATTGAGGGTTGATGATGCT
GTGTGTTTCTCGAAACTCACTAGTGTTGAGGCAGTTGCAAATTTTTCAAAAATCCCG
GCAACAATTTCTGGGGTTCGCTTTGATCAAGGGAATCATGGAGAATCACGTATCTAT
GGTAGCCCATTAGATATCACGAGGGTTAGTGGGGAATTCTCAGTGTCATTCAGAGGG
ATGAGGCTCAGACTATCTGAGATATCAGCAAGCTGCACAGGTGAGATAACAAACGT
CTCTGGTTGTTACTCCTGCATGACCGGGGCCTCAGTCAGCATAAAGTTGCATAGCAG
TAAGAACACAACAGGTCATCTTAAGTGTGATTCAGATGAGACCGCATTCAGTGTCAT
GGAGGGAACACACACATATAGGCCTCACATGAGCTTTGATAAAGCAGTAATAGATG
AGGAGTGTGTGCTAAACTGTGGTGGCCACTCATCAAAACTGCTGCTCAAAGGGAGC
CTTGTTTTCATGGACGTGCCAAGGTTTGTTGATGGGAGTTATGTCCAAACATATCAC
AGCAAGGTGCCTGCTGGGGGAAGGGTCCCAAATCCAGTAGACTGGCTCAACGCACT
GTTTGGAGATGGCATAACACGATGGATTCTTGGGATTATAGGGGTTCTGCTGGCATG
TGTCATGCTATTTGTGGTGGTGGTTGCCATCACTAGGCGATTGATCAAGGGACTGAC
TCAAAGGGCGAAGGTGGCATGATAAAGCTGGAGCCTCGGTGGCCTTGCTTCTTGCCC
CTTGGGCCTCCCCCCAGCCCCTCCTCCCCTTCCTGCACCCGTACCCCCGTGGTCTTTG
AATAAAGTCTGAGTGGGCGGCAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAATGAAGAGCATCGGATCCCGGG
CCCGTCGACTGCAGAGGCCTGCATGCAAGCTTGGTGTAATCATGGTCATAGCTGTTT
CCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCTGCATTAATGAATCGGC
CAACGCGCGGGGAGAGGCGGTTTGCGTATTGGGCGCTCTTCCGCTTCCTCGCTCACT
GACTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCAAA
AGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGA
AACCCGACAGGACTATAAAGATACCAGGCGTTTCCCCCTGGAAGCTCCCTCGTGCGC
TCTCCTGTTCCGACCCTGCCGCTTACCGGATACCTGTCCGCCTTTCTCCCTTCGGGAA
GCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAAC
GAAAACTCACGTTAAGGGATTTTGGTCATGAGATTATCAAAAAGGATCTTCACCTAG
ATCCTTTTAAATTAAAAATGAAGTTTTAAATCAAGCCCAATCTGAATAATGTTACAA
CCAATTAACCAATTCTGATTAGAAAAACTCATCGAGCATCAAATGAAACTGCAATTT
ATTCATATCAGGATTATCAATACCATATTTTTGAAAAAGCCGTTTCTGTAATGAAGG
AGAAAACTCACCGAGGCAGTTCCATAGGATGGCAAGATCCTGGTATCGGTCTGCGA
TTCCGACTCGTCCAACATCAATACAACCTATTAATTTCCCCTCGTCAAAAATAAGGT
TATCAAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCAAAAT
CACTCGCATCAACCAAACCGTTATTCATTCGTGATTGCGCCTGAGCGAGACGAAATA
CGCGATCGCTGTTAAAAGGACAATTACAAACAGGAATCGAATGCAACCGGCGCAGG
AACACTGCCAGCGCATCAACAATATTTTCACCTGAATCAGGATATTCTTCTAATACC
TGGAATGCTGTTTTTCCGGGGATCGCAGTGGTGAGTAACCATGCATCATCAGGAGTA
CGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAAC
TCTGGCGCATCGGGCTTCCCATACAAGCGATAGATTGTCGCACCTGATTGCCCGACA
TTATCGCGAGCCCATTTATACCCATATAAATCAGCATCCATGTTGGAATTTAATCGC
GGCCTCGACGTTTCCCGTTGAATATGGCTCATAACACCCCTTGTATTACTGTTTATGT
AAGCAGACAGTTTTATTGTTCATGATGATATATTTTTATCTTGTGCAATGTAACATCA
GAGATTTTGAGACACGGGCCAGAGCTGCA
15) Sequence of pUC57-Kan plasmid encoding Gn of heartland virus fused with COL1A1 signal
peptide (Gn of heartland virus fused with COL1A1 signal peptide mRNA sequence is
underlined)
SEQ ID NO: 15
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGTTCAG
CTTCGTGGACCTGAGACTGCTGCTGCTACTGGCCGCTACAGCCCTGCTGACCCACGG
CTCTCCAGGAGACCCTATTGTTTGTGGTGTGAGGACTGAAACAAACAAATCCATTCA
GATTGAGTGGAAGGAGGGGAGATCAGAGAAGCTGTGCCAGATTGACAGGCTTGGAC
ATGTCACAAGCTGGTTAAGAAACCACTCATCTTTCCAGGGGCTTATTGGTCAGGTGA
AGGGAAGACCAAGTGTTTCCTACTTCCCAGAAGGGGCTTCTTACCCAAGGTGGAGC
GGCCTATTGAGCCCATGTGATGCTGAATGGCTGGGACTGATAGCAGTGAGCAAGGC
TGGAGACACAGACATGATTGTCCCAGGCCCAACTTACAAAGGCAAAATCTTTGTTGA
GAGACCAACATACAACGGTTACAAAGGCTGGGGGTGTGCAGATGGAAAGTCACTAA
GCCACTCAGGCACATATTGTGAAACTGACAGCTCAGTGAGTTCTGGTTTAATTCAGG
GAGATAGGGTTCTCTGGGTTGGGGAAGTGGTCTGTCAGAGAGGGACCCCTGTGCCA
GAAGATGTATTTAGTGAACTGGTTAGCTTGAGTCAAAGTGAGTTCCCAGATGTGTGC
AAGATTGATGGTGTTGCATTGAACCAGTGTGAGCAGGAGAGCATCCCCCAGCCACT
GGACGTTGCATGGATTGATGTTGGAAGGTCTCATAAGGTACTGATGAGAGAACACA
AAACTAAATGGGTCCAAGAGAGCTCAGCAAAGGACTTTGTGTGCTTCAAGGTGGGT
CAGGGGCCGTGTTCAAAACAAGAGGAAGATGACTGCATGAGTAAGGGCAACTGCCA
TGGGGATGAGGTTTTCTGTAGGATGGCAGGATGCTCTGCCCGCATGCAAGATAATCA
AGAAGGCTGTAGGTGCGAACTGCTTCAAAAACCTGGAGAAATCATTGTGAATTATG
GAGGCGTCTCTGTGAGACCAACCTGTTATGGATTCTCCAGAATGATGGCAACATTGG
AAGTTCACAAACCTGATAGAGAATTAACAGGGTGCACGGGTTGTCACCTAGAGTGC
ATAGAGGGAGGAGTTAAAATTGTAACGCTTACAAGCGAGCTGAGAAGTGCAACAGT
CTGTGCTTCACACTTTTGTGCATCTGCAAAGGGGGGCTCAAAGACAACTGACATACT
CTTCCACACTGGTGCTCTCGTTGGACCCAATTCCATTAGAATAACTGGCCAGTTGTTA
GATGGGAGCAAGTTTTCCTTTGATGGGCACTGCATATTCCCAGATGGGTGCATGGCA
CTTGACTGCACCTTCTGTAAGGAGTTCCTGAGAAACCCACAATGTTACCCAGTGAAG
AAATGGCTGTTCCTGGTGGTAGTTGTAATGTGCTGCTATTGCGCCCTGATGCTGCTTA
CTAACATACTGAGAGCTATAGGTGTTTGGGGGACATGGGTTTTTGCTCCAATAAAGT
TGGCTCTAGCATTAGGGTTGAGGCTTGCCAAACTGTCAAAGAAGGGGTTGGTTGCTG
TGGTTACAAGGGGCCAAATGATCGTGAATGATGAGCTGCACCAGATTCGAGTGGAG
AGAGGTGAGCAAAATGAGGGAAGACAAGGTTGATAAAGCTGGAGCCTCGGTGGCCT
TGCTTCTTGCCCCTTGGGCCTCCCCCCAGCCCCTCCTCCCCTTCCTGCACCCGTACCC
CCGTGGTCTTTGAATAAAGTCTGAGTGGGCGGCAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAATGAAGAGCAT
CGGATCCCGGGCCCGTCGACTGCAGAGGCCTGCATGCAAGCTTGGTGTAATCATGGT
CATAGCTGTTTCCTGTGTGAAATTGTTATCCGCTCACAATTCCACACAACATACGAG
CCGGAAGCATAAAGTGTAAAGCCTGGGGTGCCTAATGAGTGAGCTAACTCACATTA
ATTGCGTTGCGCTCACTGCCCGCTTTCCAGTCGGGAAACCTGTCGTGCCAGCTGCAT
TAATGAATCGGCCAACGCGCGGGGAGAGGCGGTTTGCGTATTGGGCGCTCTTCCGCT
TCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAG
CTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTA
TCGCCACTGGCAGCAGCCACTGGTAACAGGATTAGCAGAGCGAGGTATGTAGGCGG
TGCTACAGAGTTCTTGAAGTGGTGGCCTAACTACGGCTACACTAGAAGAACAGTATT
TGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAAAA
GGATCTTCACCTAGATCCTTTTAAATTAAAAATGAAGTTTTAAATCAAGCCCAATCT
GAATAATGTTACAACCAATTAACCAATTCTGATTAGAAAAACTCATCGAGCATCAAA
TGAAACTGCAATTTATTCATATCAGGATTATCAATACCATATTTTTGAAAAAGCCGT
TTCTGTAATGAAGGAGAAAACTCACCGAGGCAGTTCCATAGGATGGCAAGATCCTG
GTATCGGTCTGCGATTCCGACTCGTCCAACATCAATACAACCTATTAATTTCCCCTCG
TCAAAAATAAGGTTATCAAGTGAGAAATCACCATGAGTGACGACTGAATCCGGTGA
GAATGGCAAAAGTTTATGCATTTCTTTCCAGACTTGTTCAACAGGCCAGCCATTACG
CTCGTCATCAAAATCACTCGCATCAACCAAACCGTTATTCATTCGTGATTGCGCCTG
AGCGAGACGAAATACGCGATCGCTGTTAAAAGGACAATTACAAACAGGAATCGAAT
GCAACCGGCGCAGGAACACTGCCAGCGCATCAACAATATTTTCACCTGAATCAGGA
TATTCTTCTAATACCTGGAATGCTGTTTTTCCGGGGATCGCAGTGGTGAGTAACCAT
GCATCATCAGGAGTACGGATAAAATGCTTGATGGTCGGAAGAGGCATAAATTCCGT
CAGCCAGTTTAGTCTGACCATCTCATCTGTAACATCATTGGCAACGCTACCTTTGCCA
TGTTTCAGAAACAACTCTGGCGCATCGGGCTTCCCATACAAGCGATAGATTGTCGCA
CCTGATTGCCCGACATTATCGCGAGCCCATTTATACCCATATAAATCAGCATCCATG
TTGGAATTTAATCGCGGCCTCGACGTTTCCCGTTGAATATGGCTCATAACACCCCTTG
TATTACTGTTTATGTAAGCAGACAGTTTTATTGTTCATGATGATATATTTTTATCTTGT
GCAATGTAACATCAGAGATTTTGAGACACGGGCCAGAGCTGCA
16) Sequence of pUC57-Kan plasmid encoding Gc of heartland virus fused with COL1A1 signal
peptide (Gc of heartland virus fused with COL1A1 signal peptide mRNA sequence is
underlined)
SEQ ID NO: 16
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGTTCAG
CTTCGTGGACCTGAGACTGCTGCTGCTACTGGCCGCTACAGCCCTGCTGACCCACGG
CTGTGATGAGCTTGTTCATGCTGAGAGTAAATCTATCACATGCAAGTCTGCATCTGG
GAATGAGAAGGAGTGCTCAGTGACAGGCAGAGCTTTGCTCCCAGCTGTTAATCCAG
GGCAGGAGGCCTGCTTGCACTTCAGCATGCCAGGAAGCCCAGACTCTAAGTGCCTC
AAGATCAAAGTGAAATCAATAAATCTCAGGTGTAAGCAAGCCTCTTCATATTATGTT
CCTGAAGCAAAGGCAAGATGTACATCTGTCAGAAGGTGCAGGTGGGCAGGTGACTG
TCAATCTGGGTGTCCAACATATTTCAGCTCGAACTCATTCTCAGATGATTGGGCAAA
CAGGATGGACAGGGCTGGGCTCGGGATGAGTGGGTGCTCAGATGGGTGTGGTGGAG
CTGCATGTGGGTGTTTCAATGCAGCGCCATCCTGCATCTTTTGGAGAAAGTGGGTGG
AGAACCCATCCAATCGTGTCTGGAAGGTGTCACCTTGTGCATCATGGGTGCTAGCTG
CAACCATTGAGTTGACCCTGCCATCAGGAGAGGTTAAGACTCTAGAGCCTGTCACAG
GGCAAGCAACTCAGATGTTCAAGGGTGTTGCAATCACATATCTGGGATCATCCATTG
AGATTGTTGGCATGACCAGGCTATGTGAGATGAAAGAGATGGGAACTGGGATAATG
GCACTAGCCCCCTGCAATGATCCAGGGCACGCCATAATGGGAAATGTGGGTGAGAT
CCAATGCAGTAGTATAGAAAGCGCAAAGCACATCAGATCTGATGGGTGCATTTGGA
ATGCTGACCTAGTTGGGATAGAATTGAGGGTTGATGATGCTGTGTGTTTCTCGAAAC
TCACTAGTGTTGAGGCAGTTGCAAATTTTTCAAAAATCCCGGCAACAATTTCTGGGG
TTCGCTTTGATCAAGGGAATCATGGAGAATCACGTATCTATGGTAGCCCATTAGATA
TCACGAGGGTTAGTGGGGAATTCTCAGTGTCATTCAGAGGGATGAGGCTCAGACTAT
CTGAGATATCAGCAAGCTGCACAGGTGAGATAACAAACGTCTCTGGTTGTTACTCCT
GCATGACCGGGGCCTCAGTCAGCATAAAGTTGCATAGCAGTAAGAACACAACAGGT
CATCTTAAGTGTGATTCAGATGAGACCGCATTCAGTGTCATGGAGGGAACACACAC
ATATAGGCCTCACATGAGCTTTGATAAAGCAGTAATAGATGAGGAGTGTGTGCTAA
ACTGTGGTGGCCACTCATCAAAACTGCTGCTCAAAGGGAGCCTTGTTTTCATGGACG
TGCCAAGGTTTGTTGATGGGAGTTATGTCCAAACATATCACAGCAAGGTGCCTGCTG
GGGGAAGGGTCCCAAATCCAGTAGACTGGCTCAACGCACTGTTTGGAGATGGCATA
ACACGATGGATTCTTGGGATTATAGGGGTTCTGCTGGCATGTGTCATGCTATTTGTG
GTGGTGGTTGCCATCACTAGGCGATTGATCAAGGGACTGACTCAAAGGGCGAAGGT
GGCATGATAAAGCTGGAGCCTCGGTGGCCTTGCTTCTTGCCCCTTGGGCCTCCCCCC
AGCCCCTCCTCCCCTTCCTGCACCCGTACCCCCGTGGTCTTTGAATAAAGTCTGAGTG
GGCGGCAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAATGAAGAGCATCGGATCCCGGGCCCGTCGACTGCAGAG
GCCTGCATGCAAGCTTGGTGTAATCATGGTCATAGCTGTTTCCTGTGTGAAATTGTTA
TCCGCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGG
TCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCT
TGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAGTT
ACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTA
AAAATGAAGTTTTAAATCAAGCCCAATCTGAATAATGTTACAACCAATTAACCAATT
CTGATTAGAAAAACTCATCGAGCATCAAATGAAACTGCAATTTATTCATATCAGGAT
TATCAATACCATATTTTTGAAAAAGCCGTTTCTGTAATGAAGGAGAAAACTCACCGA
GGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAAT
CACCATGAGTGACGACTGAATCCGGTGAGAATGGCAAAAGTTTATGCATTTCTTTCC
AGACTTGTTCAACAGGCCAGCCATTACGCTCGTCATCAAAATCACTCGCATCAACCA
AACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGC
ATCAACAATATTTTCACCTGAATCAGGATATTCTTCTAATACCTGGAATGCTGTTTTT
CCGGGGATCGCAGTGGTGAGTAACCATGCATCATCAGGAGTACGGATAAAATGCTT
GATGGTCGGAAGAGGCATAAATTCCGTCAGCCAGTTTAGTCTGACCATCTCATCTGT
AACATCATTGGCAACGCTACCTTTGCCATGTTTCAGAAACAACTCTGGCGCATCGGG
CTTCCCATACAAGCGATAGATTGTCGCACCTGATTGCCCGACATTATCGCGAGCCCA
TTTATACCCATATAAATCAGCATCCATGTTGGAATTTAATCGCGGCCTCGACGTTTCC
CGTTGAATATGGCTCATAACACCCCTTGTATTACTGTTTATGTAAGCAGACAGTTTTA
TTGTTCATGATGATATATTTTTATCTTGTGCAATGTAACATCAGAGATTTTGAGACAC
GGGCCAGAGCTGCA

### EXAMPLES

### Example 1—DNA Templates Used for In Vitro Transcription and Protein Expression

DNA template sequence for mRNA in vitro transcription (IVT) consists of T7 promoter, 5′ untranslated region (UTR), open reading frame (ORF) of glycoprotein Gn or Gc modified from the segment M glycoprotein (GenBank: MZ617372.1), 3′UTR and 120 bases of poly adenine (polyA). 5′UTR and 3′UTR are from human hemoglobin subunit alpha 1 (HBA1) mRNA (GenBank: NM_000558.5). The segment M glycoprotein was split into Gn and Gc after Gly539. The signal peptide from collagen alpha1, COL1A1 (COL1A1 SP, MFSFVDLRLLLLLAATALLTHG, GenBank: Z74615.1) was added to N-terminus of Gn and Gc ORF to facilitate its targeting. All the DNA fragments were synthesized and subcloned into pUC57-Kan vector by GenScript (Piscataway, NJ).

The Sequence of pUC57-Kan plasmid encoding Gn of Heartland virus is shown in SEQ ID NO: 13. The sequence of pUC57-Kan plasmid encoding Gc of heartland virus is shown in SEQ ID NO: 14. The sequence of pUC57-Kan plasmid encoding Gn of heartland virus fused with COL1A1 signal peptide is shown in SEQ ID NO: 15. The sequence of pUC57-Kan plasmid encoding Gc of heartland virus fused with COL1A1 signal peptide is shown in SEQ ID NO: 16.

### Example 2—In Vitro Transcription (IVT)

The plasmid vector was linearized by restriction enzyme, BspQI (New England Biolabs) for Gn and Gc forms. N1-Methylpseudouridine (m1Ψ) was purchase from BOC Sciences (Shirley, NY). IVT condition is followed by manufacture's recommendation (TranscriptAid T7 High Yield Transcription Kit, ThermoFisher) as below:
ATP/CTP/GTP/m1ψTP: 5 mM each
SmartCap (SC101, ST Pharm): 4 mM
Linear template DNA: 1 ug of plasmid
T7 RNA polymerase enzyme mix: 2 ul

IVT was carried out in 20 ul reaction incubated at 37 C for 2 hours. The template DNA is removed by 2 units of DNase I (Invitrogen) treated at 37 C for 15 min followed by a column purification (Monarch RNA Cleanup Kit, New England Biolabs).

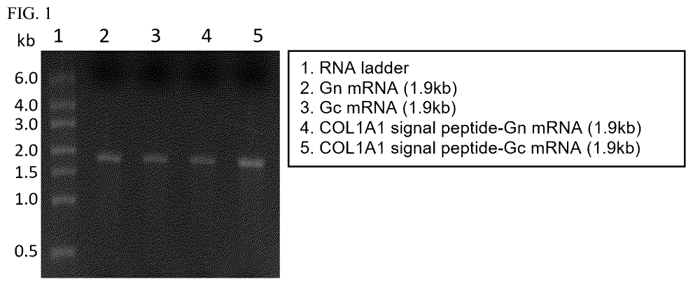

After IVT from DNA templates of HRTV mRNAs, 100 ng of mRNAs were run on 1% agarose of E-GEL EX in E-Gel Power Snap Electrophoresis Device (ThermoFisher) (one of three independent IVT products). The IVT products of four Heartland virus Gn/Gc constructs were analyzed by agarose gel, and ˜1.9 knt long mRNAs for these four mRNAs were detected as shown in FIG. 1. The IVT was conducted in triplicates.

### Example 3—Transfection

1 ug of mRNAs (synthesized in triplicates) were individually transfected into 293FT cells (Invitrogen) in 12 well plate using Lipofectamine MesseangerMax (Invitrogen), 2 ul at 1:2 ratio according to the manufacturer's protocol. Samples were collected from both media and cells after 24 hours of transfection. Cell lysates were prepared in NP-40 lysis buffer (150 mM sodium chloride/1% NP-40/50 mM Tris pH8.0). As a transfection control, 0.1 ug of EGFP mRNA (L-7601, TriLink) was co-transfected.

### Example 4—Western Blot

Rabbit anti-Heartland Virus Glycoprotein 1 antibody (#7433) for Gn and Glycoprotein 2 antibody (#7435) for Gc were purchased from ProSci Incorporated (Poway, CA). Detection of protein was using HRP-conjugated secondary antibodies (Jackson ImmunoResearch, West Grove, PA) and SuperSignal West Pico Plus Chemiluminescent Substrate (Thermo Scientific). GAPDH was detected as a loading control by HRP-conjugated mouse monoclonal antibody (sc-47724, Santa Cruz Biotechnology). EGFP, used for a mRNA transfection control, was detected by HRP-conjugated mouse monoclonal antibody (sc-9996, Santa Cruz Biotechnology).

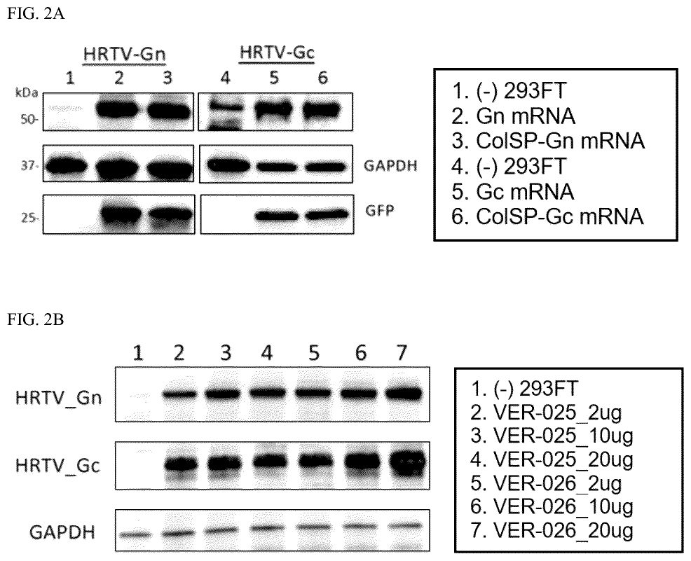

As shown in FIGS. 2A and 2B, Gn and Gc protein levels were determined by western blots. 293 FT cells or RH30 cells were individually transfected with 1 ug of the four Gn/Gc mRNAs. For Gn, both cell lysates and culture media were collected at 24 hour post transfection and subjected to Western Blot with heartland virus Gn specific antibody. For Gc, both 293 FT and RH30 cell lines were transfected, and Gc protein in the cell lysate were detected by Heartland virus Gc specific antibody. The GFP mRNA was co-transfected for a mRNA transfection control, and non-transfected 293FT cells were used as a negative control. GAPDH was used for a loading control.

Western blots were conducted from collected samples from both media for detecting any secreted proteins and cells for detecting intracellular/un-secreted proteins in 293FT or RH30 mouse muscle cell line. As shown in FIG. 2A, the Heartland virus Gn proteins both with and without COL1A1 signal (ColSP) peptide were detected in 293 FT cell lysates, but not in media. The expression of intracellular Heartland virus Gc proteins was also compared with and without COL1A1 signal peptide (ColSP) in both 293FT cells and RH30 mouse muscle cell line (FIG. 2B), and higher protein levels of Gc with COL1A1 signal peptide (ColSP) were detected, compared to Gc protein without COL1A1 signal peptide (ColSP).

### Example 5—Immunogenicity and Protection Study

This study was designed to test the neutralizing capacity as an immunogenicity in the mice of the Heartland vaccine composition of the present disclosure (e.g., Heartland vaccine compositions (1) and (2)).

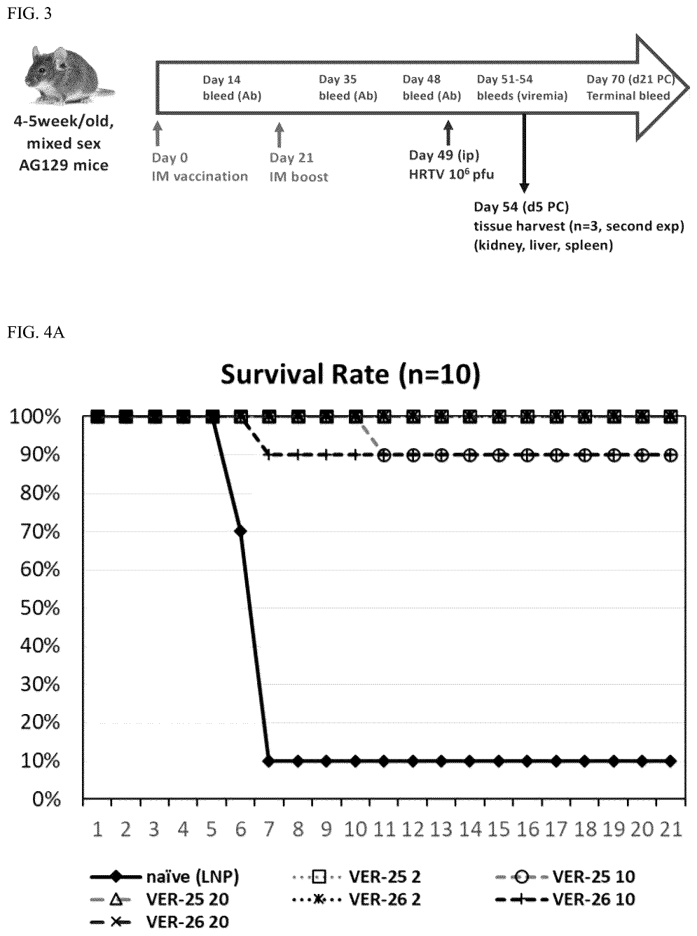

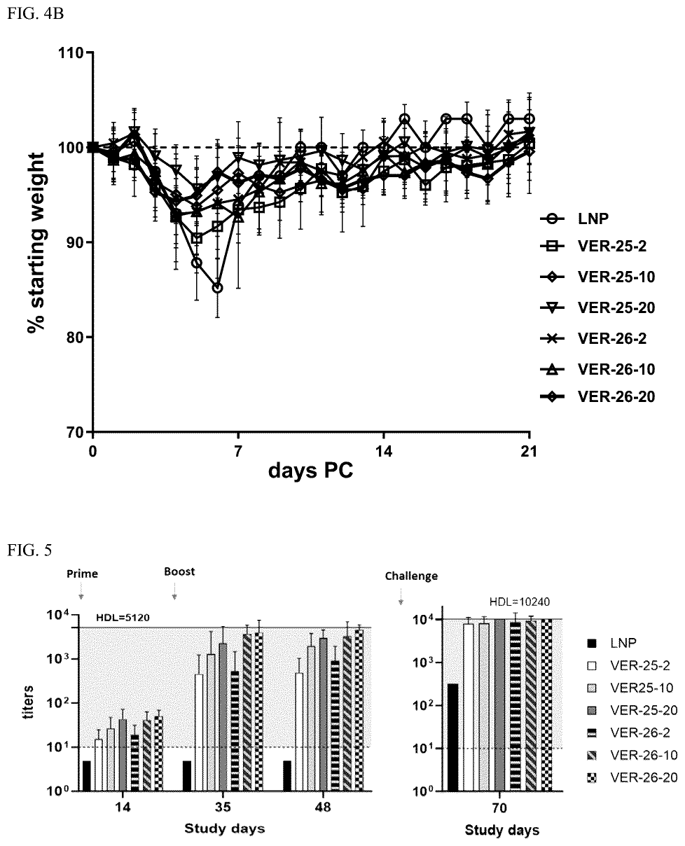

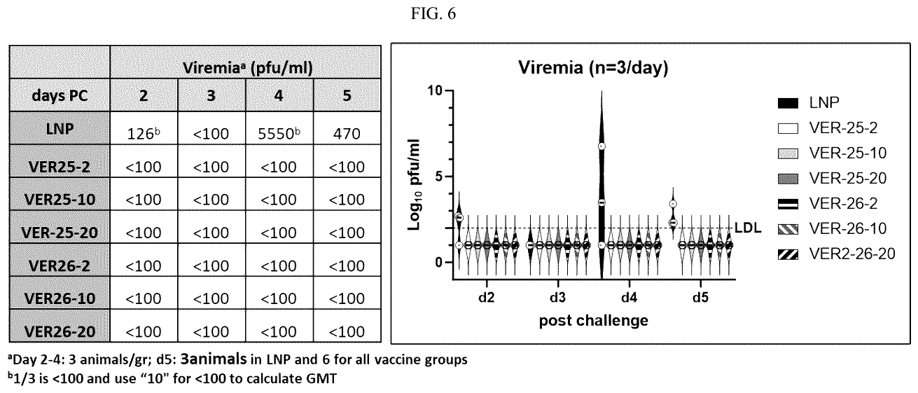

AG129 mice were immunized intramuscularly (IM) with the Heartland vaccine composition of the present disclosure (i.e, 2 ug, 10 ug, or 20 ug of mRNA formulation of Heartland vaccine compositions (1) or (2) (10 mice per dose) according to the vaccination scheme (FIG. 3). The vaccine composition of the present disclosure is chemically modified or unmodified. A total of two immunizations were given at 3-week intervals (i.e., at weeks 0, and 3), and several bloods were collected after immunization until Day 70 as shown in FIG. 3 at Day 14, Day 35, Day 48, Day 51-54 and Day 70. After a boost immunization, a lethal dose of HRTV (106 pfu) was injected intraperitoneally into the mice at day 49. All immunized mice (except for one each in VER-025 10 ug and VER-026 10 ug dose group) were protected from the lethal viral challenge while all naïve mice died (except for one) as shown in FIGS. 4A and 4B. Serum immunogenicity against HRTV for neutralizing antibodies was determined by PRNT50 (FIG. 5). After boost immunization, all the immunized mice developed strong neutralizing antibody titers against HRTV (Day 35 and Day 48 sera) in dose dependent manner as shown in FIG. 5. The neutralizing activity was further enhanced and peaked at Day 70 bleeding after viral challenge regardless of vaccine dosage. The HRTV vaccine composition (2) was higher than the Heartland vaccine composition (1) in the neutralizing activity. The HRTV viral loads in all vaccinated mice after viral challenge were below the detection limit while at least seven naïve mice displayed detectable viremia on 2, 4, or 5 days post virus challenging (FIG. 6). Both VER-025 and VER-026 mRNA vaccine compositions showed a strong immunogenicity and protection against HRTV even at lowest dose (2 ug) in the vaccinated mice proving the vaccine's efficacy.

1. A Heartland virus vaccine composition comprising
a messenger ribonucleic acid (mRNA) comprising an open reading frame (ORF) encoding Gn or Gc of Heartland virus, or the Gn or Gc of Heartland virus fused with human collagen type I alpha 1 (COL1A1) signal peptide.

2. The Heartland virus vaccine composition according to claim 1, wherein the Gn of Heartland virus has an amino acid sequence of SEQ ID NO: 1.

3. The Heartland virus vaccine composition according to claim 1, wherein the Gc of Heartland virus has an amino acid sequence of SEQ ID NO: 2.

4. The Heartland virus vaccine composition according to claim 1, wherein the Gn of Heartland virus fused with COL1A1 signal peptide has an amino acid sequence of SEQ ID NO: 3.

5. The Heartland virus vaccine composition according to claim 1, wherein the Gc of Heartland virus fused with COL1A1 signal peptide has an amino acid sequence of SEQ ID NO: 4.

6. The Heartland virus vaccine composition according to claim 1, wherein the ORF encoding Gn of Heartland virus has a nucleotide sequence of SEQ ID NO: 5.

7. The Heartland virus vaccine composition according to claim 1, wherein the ORF encoding Gc of Heartland virus has a nucleotide sequence of SEQ ID NO: 6.

8. The Heartland virus vaccine composition according to claim 1, wherein the ORF encoding Gn of Heartland virus fused with COL1A1 signal peptide has a nucleotide sequence of SEQ ID NO: 7.

9. The Heartland virus vaccine composition according to claim 1, wherein the ORF encoding Gc of Heartland virus fused with COL1A1 signal peptide has a nucleotide sequence of SEQ ID NO: 8.

10. The Heartland virus vaccine composition according to claim 1, wherein the mRNA comprising the ORF encoding Gn of Heartland virus further comprises a 5′ untranslated region (UTR), a 3′ UTR, and a poly (A) tail so as to have the following structure:
5′UTR-ORF encoding Gn of Heartland virus-3′UTR-poly (A) tail, and
wherein the ORF encoding Gn of Heartland virus has a nucleotide sequence of SEQ ID NO: 5.

11. The Heartland virus vaccine composition according to claim 1, wherein the mRNA comprising the ORF encoding Gc of Heartland virus further comprises a 5′ untranslated region (UTR), a 3′ UTR, and a poly (A) tail so as to have the following structure:
5′UTR-ORF encoding Gc of Heartland virus-3′UTR-poly (A) tail, and
wherein the ORF encoding Gc of Heartland virus has a nucleotide sequence of SEQ ID NO: 6.

12. The Heartland virus vaccine composition according to claim 1, wherein the mRNA comprising the ORF encoding Gn of Heartland virus fused with COL1A1 signal peptide further comprises a 5′ untranslated region (UTR), a 3′ UTR, and a poly (A) tail so as to have the following structure:
5′UTR-ORF encoding Gn of Heartland virus fused with COL1A1 signal peptide-3′UTR-poly (A) tail, and
wherein the ORF encoding Gn of Heartland virus fused with COL1A1 signal peptide has a nucleotide sequence of SEQ ID NO: 7.

13. The Heartland virus vaccine composition according to claim 1, wherein the mRNA comprising the ORF encoding Gc of Heartland virus fused with COL1A1 signal peptide further comprises a 5′ untranslated region (UTR), a 3′ UTR, and a poly (A) tail so as to have the following structure:
5′UTR-ORF encoding Gc of Heartland virus fused with COL1A1 signal peptide-3′UTR-poly (A) tail, and
wherein the ORF encoding Gc of Heartland virus fused with COL1A1 signal peptide has a nucleotide sequence of SEQ ID NO: 8.

14. The Heartland virus vaccine composition according to claim 10, wherein the poly (A) tail has a length of 50-250 nucleotides.

15. The Heartland virus vaccine composition according to claim 11, wherein the poly (A) tail has a length of 50-250 nucleotides.

16. The Heartland virus vaccine composition according to claim 12, wherein the poly (A) tail has a length of 50-250 nucleotides.

17. The Heartland virus vaccine composition according to claim 13, wherein the poly (A) tail has a length of 50-250 nucleotides.

18. The Heartland virus vaccine composition according to claim 10, wherein the mRNA having the structure of 5′UTR-ORF encoding Gn of Heartland virus-3′UTR-poly (A) tail has a nucleotide sequence of SEQ ID NO: 9.

19. The Heartland virus vaccine composition according to claim 11, wherein the mRNA having the structure of 5′UTR-ORF encoding Gc of Heartland virus-3′UTR-poly (A) tail has a nucleotide sequence of SEQ ID NO: 10.

20. The Heartland virus vaccine composition according to claim 12, wherein the mRNA having the structure of 5′UTR-ORF encoding Gn of Heartland virus fused with COL1A1 signal peptide-3′UTR-poly (A) tail has a nucleotide sequence of SEQ ID NO: 11.

21. The Heartland virus vaccine composition according to claim 13, wherein the mRNA having the structure of 5′UTR-ORF encoding Gc of Heartland virus fused with COL1A1 signal peptide-3′UTR-poly (A) tail has a nucleotide sequence of SEQ ID NO: 12.

22. The Heartland virus vaccine composition according to claim 10, wherein the mRNA having the structure of 5′UTR-ORF encoding Gn of Heartland virus-3′UTR-poly (A) tail has a nucleotide sequence having at least 80% identity to SEQ ID NO: 9.

23. The Heartland virus vaccine composition according to claim 11, wherein the mRNA having the structure of 5′UTR-ORF encoding Gc of Heartland virus-3′UTR-poly (A) tail has a nucleotide sequence having at least 80% identity to SEQ ID NO: 10.

24. The Heartland virus vaccine composition according to claim 12, wherein the mRNA having the structure of 5′UTR-ORF encoding Gn of Heartland virus fused with COL1A1 signal peptide-3′UTR-poly (A) tail has a nucleotide sequence having at least 80% identity to SEQ ID NO: 11.

25. The Heartland virus vaccine composition according to claim 13, wherein the mRNA having the structure of 5′UTR-ORF encoding Gc of Heartland virus fused with COL1A1 signal peptide-3′UTR-poly (A) tail has a nucleotide sequence having at least 80% identity to SEQ ID NO: 12.

26. The Heartland virus vaccine composition according to claim 1 further comprising a pharmaceutically acceptable carrier.

27. The Heartland virus vaccine composition according to claim 26, wherein the pharmaceutically acceptable carrier is a lipid nanoparticle encapsulating the mRNA therein.

28. A method of inducing immune response against Heartland virus comprising:
administering an effective amount of the Heartland virus vaccine composition according to claim 1 to a subject in need thereof.


SEQUENCE LISTING (16 sequences)

SEQ ID NO: 1 - protein, 539 aa, synthetic construct
MIVPIVLFLTLCPSELSAWGSPGDPIVCGVRTETNKSIQIEWKEGRSEKLCQIDRLGHVTSWLRNHSSFQGLIGQVKGRPSVSYFPEGASYPRWSGLLSPCDAEWLGLIAVSKAGDTDMIVPGPTYKGKIFVERPTYNGYKGWGCADGKSLSHSGTYCETDSSVSSGLIQGDRVLWVGEVVCQRGTPVPEDVFSELVSLSQSEFPDVCKIDGVALNQCEQESIPQPLDVAWIDVGRSHKVLMREHKTKWVQESSAKDFVCFKVGQGPCSKQEEDDCMSKGNCHGDEVFCRMAGCSARMQDNQEGCRCELLQKPGEIIVNYGGVSVRPTCYGFSRMMATLEVHKPDRELTGCTGCHLECIEGGVKIVTLTSELRSATVCASHFCASAKGGSKTTDILFHTGALVGPNSIRITGQLLDGSKFSFDGHCIFPDGCMALDCTFCKEFLRNPQCYPVKKWLFLVVVVMCCYCALMLLTNILRAIGVWGTWVFAPIKLALALGLRLAKLSKKGLVAVVTRGQMIVNDELHQIRVERGEQNEGRQG

SEQ ID NO: 2 - protein, 538 aa, synthetic construct
MYGPRGPIRHWLYSPALILILTTSICSGCDELVHAESKSITCKSASGNEKECSVTGRALLPAVNPGQEACLHFSMPGSPDSKCLKIKVKSINLRCKQASSYYVPEAKARCTSVRRCRWAGDCQSGCPTYFSSNSFSDDWANRMDRAGLGMSGCSDGCGGAACGCFNAAPSCIFWRKWVENPSNRVWKVSPCASWVLAATIELTLPSGEVKTLEPVTGQATQMFKGVAITYLGSSIEIVGMTRLCEMKEMGTGIMALAPCNDPGHAIMGNVGEIQCSSIESAKHIRSDGCIWNADLVGIELRVDDAVCFSKLTSVEAVANFSKIPATISGVRFDQGNHGESRIYGSPLDITRVSGEFSVSFRGMRLRLSEISASCTGEITNVSGCYSCMTGASVSIKLHSSKNTTGHLKCDSDETAFSVMEGTHTYRPHMSFDKAVIDEECVLNCGGHSSKLLLKGSLVFMDVPRFVDGSYVQTYHSKVPAGGRVPNPVDWLNALFGDGITRWILGIIGVLLACVMLFVVVVAITRRLIKGLTQRAKVA

SEQ ID NO: 3 - protein, 541 aa, synthetic construct
MFSFVDLRLLLLLAATALLTHGSPGDPIVCGVRTETNKSIQIEWKEGRSEKLCQIDRLGHVTSWLRNHSSFQGLIGQVKGRPSVSYFPEGASYPRWSGLLSPCDAEWLGLIAVSKAGDTDMIVPGPTYKGKIFVERPTYNGYKGWGCADGKSLSHSGTYCETDSSVSSGLIQGDRVLWVGEVVCQRGTPVPEDVFSELVSLSQSEFPDVCKIDGVALNQCEQESIPQPLDVAWIDVGRSHKVLMREHKTKWVQESSAKDFVCFKVGQGPCSKQEEDDCMSKGNCHGDEVFCRMAGCSARMQDNQEGCRCELLQKPGEIIVNYGGVSVRPTCYGFSRMMATLEVHKPDRELTGCTGCHLECIEGGVKIVTLTSELRSATVCASHFCASAKGGSKTTDILFHTGALVGPNSIRITGQLLDGSKFSFDGHCIFPDGCMALDCTFCKEFLRNPQCYPVKKWLFLVVVVMCCYCALMLLTNILRAIGVWGTWVFAPIKLALALGLRLAKLSKKGLVAVVTRGQMIVNDELHQIRVERGEQNEGRQG

SEQ ID NO: 4 - protein, 532 aa, synthetic construct
MFSFVDLRLLLLLAATALLTHGCDELVHAESKSITCKSASGNEKECSVTGRALLPAVNPGQEACLHFSMPGSPDSKCLKIKVKSINLRCKQASSYYVPEAKARCTSVRRCRWAGDCQSGCPTYFSSNSFSDDWANRMDRAGLGMSGCSDGCGGAACGCFNAAPSCIFWRKWVENPSNRVWKVSPCASWVLAATIELTLPSGEVKTLEPVTGQATQMFKGVAITYLGSSIEIVGMTRLCEMKEMGTGIMALAPCNDPGHAIMGNVGEIQCSSIESAKHIRSDGCIWNADLVGIELRVDDAVCFSKLTSVEAVANFSKIPATISGVRFDQGNHGESRIYGSPLDITRVSGEFSVSFRGMRLRLSEISASCTGEITNVSGCYSCMTGASVSIKLHSSKNTTGHLKCDSDETAFSVMEGTHTYRPHMSFDKAVIDEECVLNCGGHSSKLLLKGSLVFMDVPRFVDGSYVQTYHSKVPAGGRVPNPVDWLNALFGDGITRWILGIIGVLLACVMLFVVVVAITRRLIKGLTQRAKVA

SEQ ID NO: 5 - RNA, 1617 nt, synthetic construct
  source 1..1617: mol_type = other RNA
atgatcgtgcccattgtcctgtttctcacgctctgtccgtccgaactcagtgcctggggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggt

SEQ ID NO: 6 - RNA, 1614 nt, synthetic construct
  source 1..1614: mol_type = other RNA
atgtacggacctagaggccccattcgtcactggctatactcacctgcccttattctcattctcaccacttcaatttgctctggatgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggca

SEQ ID NO: 7 - RNA, 1623 nt, synthetic construct
  source 1..1623: mol_type = other RNA
atgttcagcttcgtggacctgagactgctgctgctactggccgctacagccctgctgacccacggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggt

SEQ ID NO: 8 - RNA, 1596 nt, synthetic construct
  source 1..1596: mol_type = other RNA
atgttcagcttcgtggacctgagactgctgctgctactggccgctacagccctgctgacccacggctgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggca

SEQ ID NO: 9 - RNA, 1905 nt, synthetic construct
  source 1..1905: mol_type = other RNA
aggccggcactcttctggtccccacagactcagagagaacccgccgccaccatgatcgtgcccattgtcctgtttctcacgctctgtccgtccgaactcagtgcctggggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggttgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 1902 nt, synthetic construct
  source 1..1902: mol_type = other RNA
aggccggcactcttctggtccccacagactcagagagaacccgccgccaccatgtacggacctagaggccccattcgtcactggctatactcacctgcccttattctcattctcaccacttcaatttgctctggatgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - RNA, 1911 nt, synthetic construct
  source 1..1911: mol_type = other RNA
aggccggcactcttctggtccccacagactcagagagaacccgccgccaccatgttcagcttcgtggacctgagactgctgctgctactggccgctacagccctgctgacccacggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggttgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 1884 nt, synthetic construct
  source 1..1884: mol_type = other RNA
aggccggcactcttctggtccccacagactcagagagaacccgccgccaccatgttcagcttcgtggacctgagactgctgctgctactggccgctacagccctgctgacccacggctgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 4509 bp, synthetic construct
  source 1..4509: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgatcgtgcccattgtcctgtttctcacgctctgtccgtccgaactcagtgcctggggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggttgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 14 - DNA, 4506 bp, synthetic construct
  source 1..4506: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgtacggacctagaggccccattcgtcactggctatactcacctgcccttattctcattctcaccacttcaatttgctctggatgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 15 - DNA, 4515 bp, synthetic construct
  source 1..4515: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgttcagcttcgtggacctgagactgctgctgctactggccgctacagccctgctgacccacggctctccaggagaccctattgtttgtggtgtgaggactgaaacaaacaaatccattcagattgagtggaaggaggggagatcagagaagctgtgccagattgacaggcttggacatgtcacaagctggttaagaaaccactcatctttccaggggcttattggtcaggtgaagggaagaccaagtgtttcctacttcccagaaggggcttcttacccaaggtggagcggcctattgagcccatgtgatgctgaatggctgggactgatagcagtgagcaaggctggagacacagacatgattgtcccaggcccaacttacaaaggcaaaatctttgttgagagaccaacatacaacggttacaaaggctgggggtgtgcagatggaaagtcactaagccactcaggcacatattgtgaaactgacagctcagtgagttctggtttaattcagggagatagggttctctgggttggggaagtggtctgtcagagagggacccctgtgccagaagatgtatttagtgaactggttagcttgagtcaaagtgagttcccagatgtgtgcaagattgatggtgttgcattgaaccagtgtgagcaggagagcatcccccagccactggacgttgcatggattgatgttggaaggtctcataaggtactgatgagagaacacaaaactaaatgggtccaagagagctcagcaaaggactttgtgtgcttcaaggtgggtcaggggccgtgttcaaaacaagaggaagatgactgcatgagtaagggcaactgccatggggatgaggttttctgtaggatggcaggatgctctgcccgcatgcaagataatcaagaaggctgtaggtgcgaactgcttcaaaaacctggagaaatcattgtgaattatggaggcgtctctgtgagaccaacctgttatggattctccagaatgatggcaacattggaagttcacaaacctgatagagaattaacagggtgcacgggttgtcacctagagtgcatagagggaggagttaaaattgtaacgcttacaagcgagctgagaagtgcaacagtctgtgcttcacacttttgtgcatctgcaaaggggggctcaaagacaactgacatactcttccacactggtgctctcgttggacccaattccattagaataactggccagttgttagatgggagcaagttttcctttgatgggcactgcatattcccagatgggtgcatggcacttgactgcaccttctgtaaggagttcctgagaaacccacaatgttacccagtgaagaaatggctgttcctggtggtagttgtaatgtgctgctattgcgccctgatgctgcttactaacatactgagagctataggtgtttgggggacatgggtttttgctccaataaagttggctctagcattagggttgaggcttgccaaactgtcaaagaaggggttggttgctgtggttacaaggggccaaatgatcgtgaatgatgagctgcaccagattcgagtggagagaggtgagcaaaatgagggaagacaaggttgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 16 - DNA, 4488 bp, synthetic construct
  source 1..4488: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgttcagcttcgtggacctgagactgctgctgctactggccgctacagccctgctgacccacggctgtgatgagcttgttcatgctgagagtaaatctatcacatgcaagtctgcatctgggaatgagaaggagtgctcagtgacaggcagagctttgctcccagctgttaatccagggcaggaggcctgcttgcacttcagcatgccaggaagcccagactctaagtgcctcaagatcaaagtgaaatcaataaatctcaggtgtaagcaagcctcttcatattatgttcctgaagcaaaggcaagatgtacatctgtcagaaggtgcaggtgggcaggtgactgtcaatctgggtgtccaacatatttcagctcgaactcattctcagatgattgggcaaacaggatggacagggctgggctcgggatgagtgggtgctcagatgggtgtggtggagctgcatgtgggtgtttcaatgcagcgccatcctgcatcttttggagaaagtgggtggagaacccatccaatcgtgtctggaaggtgtcaccttgtgcatcatgggtgctagctgcaaccattgagttgaccctgccatcaggagaggttaagactctagagcctgtcacagggcaagcaactcagatgttcaagggtgttgcaatcacatatctgggatcatccattgagattgttggcatgaccaggctatgtgagatgaaagagatgggaactgggataatggcactagccccctgcaatgatccagggcacgccataatgggaaatgtgggtgagatccaatgcagtagtatagaaagcgcaaagcacatcagatctgatgggtgcatttggaatgctgacctagttgggatagaattgagggttgatgatgctgtgtgtttctcgaaactcactagtgttgaggcagttgcaaatttttcaaaaatcccggcaacaatttctggggttcgctttgatcaagggaatcatggagaatcacgtatctatggtagcccattagatatcacgagggttagtggggaattctcagtgtcattcagagggatgaggctcagactatctgagatatcagcaagctgcacaggtgagataacaaacgtctctggttgttactcctgcatgaccggggcctcagtcagcataaagttgcatagcagtaagaacacaacaggtcatcttaagtgtgattcagatgagaccgcattcagtgtcatggagggaacacacacatataggcctcacatgagctttgataaagcagtaatagatgaggagtgtgtgctaaactgtggtggccactcatcaaaactgctgctcaaagggagccttgttttcatggacgtgccaaggtttgttgatgggagttatgtccaaacatatcacagcaaggtgcctgctgggggaagggtcccaaatccagtagactggctcaacgcactgtttggagatggcataacacgatggattcttgggattataggggttctgctggcatgtgtcatgctatttgtggtggtggttgccatcactaggcgattgatcaagggactgactcaaagggcgaaggtggcatgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca